(19) United States
(12) Patent Application Publication
(10) Pub. No.: US 2024/0207454 A1
(43) Pub. Date: Jun. 27, 2024

(54) NEURO-BEHAVIORAL ASSAY FOR PHYSIOLOGICAL EVALUATION OF WATER-SOLUBLE ANESTHETICS

(71) Applicant: Northeastern University, Boston, MA (US)

(72) Inventors: Gunther Zupanc, Auburndale, MA (US); David Lehotzky, Boston, MA (US)

(21) Appl. No.: 18/538,612

(22) Filed: Dec. 13, 2023

Related U.S. Application Data
(60) Provisional application No. 63/432,150, filed on Dec. 13, 2022.

(51) Int. Cl. A61K 49/00

(52) U.S. Cl. CPC A61K 49/0008

(57) ABSTRACT

Disclosed are a system and an in vivo assay for the physiological evaluation of water-soluble general anesthetics. The assay is based on the effects of anesthetics on the frequency of the pacemaker nucleus, an endogenous neural oscillator in the brainstem, of the weakly electric fish Apteronotus leptorhynchus.

### RELATED APPLICATIONS

This application claims the benefit of priority to U.S. Provisional Patent Application No. 63/432,150, filed Dec. 13, 2022.

### GOVERNMENT SUPPORT

This invention was made with government support under Grant Number 1946910 awarded by the National Science Foundation. The government has certain rights in the invention.

### BACKGROUND

There is significant need for development of water-soluble general anesthetics because they have several advantages compared to lipid-soluble general anesthetics. The application of water-soluble anesthetics is particularly preferential for elderly (people 65 or older). Since they have increased adipose tissue and decreased body water, the volume of distribution of lipid-soluble anesthetics increases in elderly patients, which retards the elimination of these agents and prolongs their half-life. At the same time, elderly people are more sensitive to the most widely used lipid-soluble general anesthetic, propofol, an effect that correlates with their increased vulnerability to delirium and cognitive decline in the postoperative period. These complications, in turn, are associated with increased mortality.

For the development of novel water-soluble anesthetics, the availability of assays for screening of suitable candidate molecules is instrumental. While assays for chemotypic high-throughput screening are currently being developed, and assays for behavioral screening of novel anesthetic compounds are already available, high-throughput assays that evaluate the neurophysiological effects, particularly at the systems level, are missing.

### SUMMARY OF THE INVENTION

The assay described herein has great potential to fill the gap between chemotypic screening and behavioral screening by providing an assay whose setup costs are only a fraction of the costs for establishing one of the traditional physiological setups. Equally important, the assay produces high-quality results much faster than any of the traditional assays based on in vivo or in vitro physiological recording.

An in vivo assay for physiological evaluation of water-soluble general anesthetics has been developed. This assay is based on the effects of anesthetics on the frequency of the pacemaker nucleus, an endogenous neural oscillator in the brainstem, of the weakly electric fish Apteronotus leptorhynchus. Alterations of its frequency are determined using the fish's electric organ discharge (EOD) as a readily available proxy. The effects of anesthetics on the EOD frequency can be utilized to screen compounds for possible anesthetic properties, and to evaluate how such active compounds affect neural activity in the central nervous system at a systems level.

This disclosure describes the setup of the assay and outlines the algorithm used for automatic measurement of the EOD frequency, including transient modulations. The assay's applicability is illustrated by showing the effect of three water-soluble anesthetics—urethane, MS-222, and eugenol—on the EOD frequency. Finally, the advantages of the described assay compared to other screening approaches is described.

### BRIEF DESCRIPTION OF THE DRAWINGS

FIG. 1 Experimental setup for recording of the EOD. Modified after Sîrbulescu et al. 2009.

FIG. 2 Flow diagram outlining the design of Experiment 1.

FIG. 3 Effect of urethane anesthesia on EOD frequency. The frequency was determined at 1-min intervals. To establish the baseline frequency, the EOD was recorded in the fish's home tank in both the handling experiments (blue diamonds) and the urethane-anesthesia experiments (red circles). For anesthesia, the fish was transferred to a beaker with a 2.5% urethane solution in aquarium water. As soon as the fish stopped undulating its anal fin and moving its opercula, it was returned to the home tank (arbitrarily defined as time point ‘0’). The time the fish was in the urethane solution is indicated by the gray bar. As a control, the fish was handled identically, but transferred to a beaker filled with aquarium water only, where it spent a similar amount of time to that of fish exposed to the anesthetic. In each of the 8 fish, the urethane-anesthesia experiments and the handling experiments were conducted on 2 different days. Note that the baseline EODs of the 8 individual fish cover a wide range of frequencies, including those typical of females (low frequencies) and males (high frequencies).

FIG. 4 Time course of frequency change during anesthesia with urethane (a) and MS-222 (b). The frequency was determined at 1-s intervals. After 10 min of baseline recording, the tube with the fish was transferred from the home tank to a smaller tank with a 2.5% solution of urethane or a 0.02% solution of MS-222, both in aquarium water from the fish's home tank. Recording continued during the fish's exposure to the anesthetic. After anesthesia, the fish tube was transferred back to the home tank. The gray bar indicates the time fish was exposed to the respective anesthetic. The beginning of anesthesia is arbitrarily defined as time point ‘0’.

FIG. 5 Effect of MS-222 anesthesia on EOD frequency. The frequency was determined at 1-min intervals. To establish the baseline frequency, the EOD was recorded in the fish's home tank in both the handling experiments (blue diamonds) and the MS-222-anesthesia experiments (red circles). For anesthesia, the fish was transferred to a beaker with a 0.02% MS-222 solution in aquarium water. As soon as the fish stopped undulating its anal fin and moving its opercula, it was returned to the home tank (arbitrarily defined as time point ‘0’). The time the fish was in the MS-222 solution is indicated by the gray bar. As a control, the fish was handled identically, but transferred to a beaker filled with only aquarium water, where it spent a similar amount of time to that of fish exposed to the anesthetic. In each of the 8 fish, the MS-222-anesthesia and the handling experiments were conducted on 2 different days.

FIG. 6 Effect of urethane (a) and MS-222 (b) anesthesia on the ratio of EOD frequency vs. baseline frequency. This analysis is based on the EOD recordings shown in FIGS. 3 and 5, respectively. To establish the baseline frequency, the median frequency of the 30-min EOD recording prior to exposure to the respective anesthetic was used. The return of the fish to its isolation tank is arbitrarily defined as time point ‘0’. Data points for all 8 fish (red circles) are shown together with the 4-parameter model fitted using robust nonlinear regression (black curve).

FIG. 7 Effect of urethane anesthesia on chirping behavior. Chirp detection was based on the same EOD recordings that were used for the time-frequency plots shown in FIG. 3. The time during which the fish was exposed to urethane is indicated by the gray bar. Note the extremely low number of spontaneously generated chirps throughout the handling experiment (blue bars) and during the initial baseline recording in the urethane experiment (red bars). Urethane anesthesia resulted in a powerful transient increase in chirp production.

FIG. 8 Temporal dynamics of changes in chirp rate during urethane anesthesia (a) and MS-222 anesthesia (b). The fish's EOD was recorded continuously throughout the experiment. After establishment of the baseline spontaneous activity over the first 10 min, the fish was anesthetized in 2.5% urethane or 0.02% MS-222 solution in aquarium water (light gray arca; timepoint ‘0’ marks the beginning of anesthesia), followed by a few extra minutes post anesthesia. Note absence of spontaneous chirps during the baseline EOD recording of the second fish (middle row).

FIG. 9 Effect of MS-222 anesthesia on chirping behavior. Chirp detection was based on the same EOD recordings that were used for the time-frequency plots shown in FIG. 5. The time during which the fish was exposed to MS-222 is indicated by the gray bar. Chirp production was elevated after MS-222 anesthesia (red bars), compared to the number of chirps observed in the handling experiment (blue bars) or during the initial baseline recording. However, this increase was far less pronounced than in the urethane experiment (cf. FIG. 7). Note the difference in scaling of the y-axes between the two figures.

FIG. 10 Frequency shape of chirps normalized by peak frequency rise used for chirp detection as described. During chirping, the time course of normalized frequency was modeled by a single-parameter function Φ(ξ;α)(see Eq. 2). Time is denoted by ξ and different colors correspond to different shape parameter values α.

FIG. 11 Flow diagram outlining the experimental design.

FIG. 12 Effect of eugenol anesthesia on EOD frequency. The frequency was determined at 30-s intervals through a pair of electrodes built into a shelter tube. To establish the baseline frequency, the EOD was recorded in the fish's home tank for 30 min. Then, the shelter tube with the fish was transferred to a smaller tank containing aquarium water from the home tank(control: blue diamonds) or eugenol in aquarium water at one of three concentrations (30 μL/L: orange circles; 45 μL/L: green squares; 60 μL/L: red crosses). After 5 min (indicated by the gray bar), the fish was returned to its home tank, where recording of the EOD continued for another 180 min. The return of the fish to its home tank was arbitrarily defined as time point ‘0’. Each of the 4 experiments was conducted on a different day. Note that the baseline EODs of the 8 individual fish cover a wide range of frequencies, including those typical of females (low frequencies) and males (high frequencies). The plots of the individual fish are presented in ascending order according to their baseline EOD frequency.

FIG. 13 Dose-dependent effect of eugenol on EOD frequency. The plot is based on the EOD recordings shown in FIG. 4. For the analysis, the minimum value of median EOD frequencies determined at 30-s intervals was related to the median EOD frequency determined during the 30 min immediately preceding the 5 min of the fish's exposure to the anesthetic solution. For the control experiment (blue) and each of the 3 eugenol concentrations (30 μl/L: orange; 45 μl/L: green; 60 μl/L: red), the normalized maximum frequency drops exhibited by each of the 8 fish are shown (diamonds, circles, squares, and crosses, respectively), together with the medians (bars) of these values.

FIG. 14 Recovery of EOD frequency after eugenol anesthesia. The graph is based on the EOD recordings shown in FIG. 4. The recovery is characterized by the EOD frequencies normalized to the median frequency of the 30-min EOD recording prior to the fish's transfer to the small tank with water from its home tank (control: blue diamonds) or one of the three concentrations of eugenol in aquarium water (30 μl/L: orange circles;45 μl/L: green squares; 60 μl/L: red crosses). The return of the fish to its home tank is arbitrarily defined as time point ‘0’. Data points for all 8 fish are shown together with the 4-parameter model fitted using nonlinear regression (black curves). The fitted model parameters are summarized in Table 2. The timepoint of recovery is indicated with vertical black dashed lines.

FIG. 15 Effect of eugenol anesthesia at a concentration of 30 μL/L on the generation of type-2 chirps. Chirp detection was based on the same EOD recordings that were used for the time-frequency plots shown in FIG. 4. Eugenol anesthesia for 5 min (indicated by the gray bar) induced an increase in the rate of chirping (orange bars), particularly during the 30 min following the fish's exposure to eugenol, compared to the chirp rates prior to anesthesia or during the control experiments (blue bars). Note inter-individual differences in the effect of eugenol on the number of chirps produced.

FIG. 16 Effect of eugenol anesthesia at a concentration of 45 μL/L on the generation of type-2 chirps. Eugenol anesthesia for 5 min (indicated by the gray bar) induced an increase in the rate of chirping (green bars), particularly during the 30 min following the fish's exposure to eugenol, compared to the chirp rate prior to anesthesia or the control experiments (blue bars).

FIG. 17 Effect of eugenol anesthesia at a concentration of 60 μL/L on the generation of type-2 chirps. Eugenol anesthesia for 5 min (indicated by the gray bar) induced an increase in the rate of chirping (red bars), compared to pre-anesthesia and control levels (blue bars).

FIG. 18 Effect of eugenol anesthesia on the number of type-2 chirps. The change in the number of type-2 chirps between the 30 min interval preceding the fish's exposure to the anesthetic and the 30 min interval after its exposure was determined for the control experiment (blue) and each of the 3 eugenol concentrations (30 μl/L: orange; 45 μl/L: green; 60 μl/L: red) for each of the 8 fish (diamonds, circles, squares, and crosses, respectively). For the control and the three eugenol concentrations, the median values are indicated with bars.

FIG. 19 Effect of eugenol anesthesia on the number of type-2 chirps. The change in the number of type-2 chirps between the 30 min interval preceding the fish's exposure to the anesthetic and the 30 min interval after its exposure was determined for the control experiment (blue) and each of the 3 eugenol concentrations (30 μl/L: orange; 45 μl/L: green; 60 μl/L: red) for each of the 8 fish (diamonds, circles, squares, and crosses, respectively). For the control and the three eugenol concentrations, the median values are indicated with bars of the shelter tube. Approximately 1.5 min after the onset of the anesthesia, the fish's movements ceased, and, thus, the jit-tering of the EOD amplitude became minimal. Approximately 3.5 min after the start of the anesthesia, the EOD amplitude collapsed to very low levels. Within 1 min of return of the fish to its home tank, the EOD amplitude gradually recovered. It reached baseline levels approximately 6 min after the end of the fish's exposure to the eugenol solution.

FIG. 20 EOD frequency f (a) and amplitude A (b) with respect to time T in a recording involving urethane anesthesia. After baseline recording, the tube with the fish was transferred from the home tank to a glass beaker containing 2.5% urethane solution dissolved in aquarium water. As soon as the fish stopped undulating its anal fin and moving its opercula, it was returned to the home tank (arbitrarily defined as time point T=0). The gray bar indicates the time during which the fish was exposed to the anesthetic. Changes in the orientation and position of the fish relative to the recording electrodes result in noisy amplitude signals (pre-anesthesia, and T>˜2000 s as shown in b). The reduction of noise immediately after anesthesia is related to the ceased movement of the fish. Note onset of type-2 chirping at higher rates immediately after anesthesia (a/A1, b/B1) that persists to approximately T=4600 s after exposure to the anesthetic (a/A2, b/B2). The recorded signal contains both type-2 (a/A1′, b/B1′) and type-1 (a/A2′, b/B2′) chirps. The latter is characterized by large rise and negative undershoot in frequency (a/A2′), as well as a large drop in amplitude (b/B2′). By contrast, the former is characterized by a smaller rise without undershoot in frequency (a/A1′) and a smaller reduction in amplitude (b/B1′).

FIG. 21 Matlab tool built for collecting chirp samples from time-series frequency data (black dots). The user can select data points associated with a chirp by moving the cursor (intersection of black lines in a and c) to the two end points of the time interval delimiting the chirp instance. After selecting the time interval (red lines in b), the user must confirm the current selection before proceeding to collect further data points (see dialogue box in b). Following the confirmation of the selection, data points associated with the selected time interval are stored and removed from the displayed data set (c). Once all displayed chirp instances have been collected, the user can move to the next (or previous), overlapping, time segment to collect the remaining chirp data points from the time-series frequency data segment.

FIG. 22 Processing of “ground truth” samples. Data points {(T_{i,j}, f_{i,j})}}ⱼ₌₁^{li} of the i-th sample are plotted in a as black dots. The frequency values {f_{i,j}}ⱼ₌₁^{li} are normalized according to Eq. 14 and passed through the rectifier function (red curve) displayed in b. The green dashed lines in b and c display the cutoff value −φi of the rectifier function. The centered and normalized data points {(˜T_{i,j}, φ_{i,j})}ⱼ₌₁^{li} of the i-th sample (see Eqs. 14-20) are displayed in c as black dots together with the rectified normalized frequencies (red curve) and their empirical cumulative distribution (blue curve). The 10% and 90% percentile estimates (blue, dashed lines in c) of this cumulative distribution determine the time width of the sample: Δ˜Tᵢ=˜T_{i,j}+−˜T_{i,j-i}. The sample is trimmed based on this time width (d) such that data points outside interval ˜T_{i,j}∈[−3Δ˜Tᵢ, 3 Δ˜Tᵢ] (delimited by black, dashed lines and marked by gray dots) are eliminated.

FIG. 23 Training data set projected to the space spanned by the first two principal components (PC1 and PC2). Circles with different colors correspond to clusters identified by the algorithm. Gray crosses correspond to samples in an eliminated cluster. The percentage-wise size of kept (circles) and eliminated (crosses) clusters are indicated at the top-left corner, relative to the size of the training set.

FIG. 24 Illustration of the chirp detection methods described. Different rows correspond to different time instants (t₁<t₂<t₃<t₄) of the “sliding” time window indicated by vertical green lines in a. At each time instant, the Mahalanobis-distance-based detection algorithm (b) normalizes the data set inside the time window (green crosses in a) and projects it to the space spanned by the principal components of the training set (green cross in b). If the squared Mahalanobis distance value d2 associated with this projected point (indicated at the top of each row in b) is below the limit of the cluster with highest posterior probability (corresponding to the color-coded ellipse in b), then the Mahalanobis-distance-based algorithm may detect a chirp (2nd and 3rd row). At each time instant, the coefficient-of-determination-based algorithm (c) normalizes the data set inside the time window and computes its coefficient of determination with respect to each cluster mean. The highest coefficient-of-determination value R² among all cluster means is indicated at the top of each row in c, and the related cluster mean is plotted (color-coded line in c). If this value is above a threshold, then the coefficient-of-determination-based algorithm may detect a chirp.

FIG. 25 Time-frequency shape function used for chirp detection in Eske et al (2023). The time course of normalized frequency (sec Eq. 14) during chirping is modeled by a single-parameter function φ(˜T; α). Different colors correspond to different shape parameter values α.

FIG. 26 Lowest achievable average false negative rate (r_{FN}) as a function of maximum allowed average false positive rate (r_{FP}), using k-fold cross validation with k=2 (a) and k=4 (b). Curves were calculated for “ground truth” data set G2 according to Section 2.5. Different colors are associated with different methods, as indicated in the top-right corner of b.

FIG. 27 Normalized frequency (a) and amplitude (b) in the first two principal components (PC1 and PC2) of the “ground truth” data set G₂. Explained variance as a function of retained principal components (gray line in c). The black cross in c corresponds to optimal hyper-parameter settings hopt(rFP=5%) determined by 4-fold cross validation.

FIG. 28 Normalized frequency (a) and amplitude (b) components of cluster means for the model trained on the entire “ground truth” data set G₂. Clusters were computed under optimal parameter settings hₒₚₜ(r_{FP}=5%) determined via 4-fold cross validation. Relative voltage amplitude decrease−maximum frequency rise pairs plotted for each sample (c) reveal that G₂ consists entirely of type-2 chirps. Different colors are associated with different clusters, their proportions are indicated in the top-right corner of a.

FIG. 29 Cluster mean (red) and detected chirp samples (gray) in a sub-cluster (containing 264 samples) related to the cluster with 6.73% proportion in FIG. 9a. The black curve displays the median of detected chirp samples across feature dimensions.

FIG. 30 Locomotor activity inferred from amplitude fluctuations of the EOD. After recording of the baseline EOD for 6 min, the fish was transferred to a 0.02% MS-222 solution in aquarium water for 5 min. After return of the fish to its home tank, the EOD recording continued for 15 min. To determine the locomotor activity index, positive and negative deviations from an arbitrarily set threshold value of the amplitude were determined every 1 sec, and the standard deviation of the absolute values of these amplitude deviations were calculated for 30-sec time windows. The graph shows that within 2 min of the start of the fish's 5 min exposure to the anesthetic (open circles), the locomotor activity drops dramatically, compared to baseline levels (filled circles). Recovery of the locomotor activity occurred within some 2 min after the fish was returned to its home tank (filled circles).

FIG. 31 Effect of the water-soluble anesthetic MS-222 on the synchronized oscillation frequency of the isolated pacemaker nucleus in the weakly electric fish Apteronoptus leptorhynchus. The pacemaker nucleus was continuously flushed with artificial cerebrospinal fluid (ACSF) to which MS-222 at various concentrations was added (A=0.001%, B=0.005%, C=0.01%, D=0.02%, E=0.03%, F=0.04%, G=0.04%, H=0.05%, I=0.06%; duration in all stimulations for 2 min, except in F, where the nucleus was stimulated for 4 min). Exposure of the in vitro preparation of the pacemaker nucleus leads within a few seconds to a dramatic decrease in the frequency of the synchronized oscillations of the neurons of the pacemaker nucleus. Note that at the highest concentrations of MS-222 (F-I), the amplitude of the neural oscillations collapses after the drop in frequency. However, in each case the oscillation frequency recovers after switching the superfluent back for the MS-222/ACSF to ACSF only.

### DETAILED DESCRIPTION

The problem of physiological screening of water-soluble general anesthetics is addressed. Instead of recording neural activity directly from a specific site in the central nervous system, a behavioral proxy of such activity was us. This is possible because the system on which the assay is based (an endogenous brainstem oscillator called the pacemaker nucleus) drives the behavioral activity (the electric organ discharge of the electric fish) in a one-to-one fashion. As a result, the frequencies of the endogenous neural oscillator and the electric organ discharge are identical. Changes in the frequency of the pacemaker nucleus induced by anesthetics can, thus, be evaluated by monitoring the frequency of the electric organ discharge.

The evaluation of the effect of anesthetic compounds on the neural activity of a structure in the central nervous system by using a behavioral proxy, instead of directly recording the neural activity from this structure, makes the application of the assay relatively simple while producing high-quality results more reliably and in much shorter time than possible by using physiological approaches. Moreover, the costs for the setup of the assay are only about 10% of the costs for establishing a setup for direct in vivo or in vitro physiological recording.

The assay described herein delivers results with high reliability (almost every run yields high-quality experimental data) and high reproducibility (runs using different animals but same anesthetics yield data that are fundamentally very similar, whereas runs using the same animal but different anesthetics yield data that indicate fundamental differences in the effects of different drugs). In vivo and in vitro neurophysiological assays notoriously suffer from failures, resulting in low yield.

There is a need for general anesthetics with improved safety and specificity. In particular, the traditional view that anesthesia depends on lipid solubility of anesthetic agents has been shown to be a misunderstanding of experimental data. Lipophilic anesthetics require formulations in lipid-containing solvents, which bears several serious disadvantages. For example, propofol, one of the most widely employed general anesthetics, is a lipid-based emulsion formulation of the active drug diisopropyl phenol. This emulsion tends to separate when other chemicals are added or when temperature and pH change. The lipid-based emulsion of propofol also contributes to hyperlipidemia, bears the risk of infection by bacterial contamination, and causes pain on injection. Moreover, the soybean oil and egg lecithin solubilizing agents are potential allergens.

The application of water-soluble anesthetics can be useful for elderly (people 65 or older). Since they have increased adipose tissue and decreased body water, the volume of distribution of lipid-soluble anesthetics increases in elderly patients, which retards the elimination of these agents and prolongs their half-life (for review see Rivera and Antognini 2009). At the same time, elderly people are more sensitive to propofol, an effect that correlates with their increased vulnerability to delirium and cognitive decline in the postoperative period. These complications, in turn, are associated with increased mortality.

Anesthetics preferably are water soluble. Efforts for developing such drugs have traditionally focused on modifying the chemical structure, and thereby the activity, of existing drugs. Only in recent years have unbiased high-throughput approaches been established to screen large libraries of compounds for anesthetic ligands. Such chemotypic screens have been complemented by high-throughput behavioral screening, such as the Xenopus tadpole assay, to validate the anesthetic properties of the identified ligand. A high-throughput physiological approach to evaluate anesthetic ligand candidates at a systems level was needed. Existing electrophysiological approaches can be designed for examining the neurophysiological effect of anesthetics in vitro at the levels of ion channels of single cells but not at the systems level. Moreover, the application of these assays can be time-intensive and expensive, and they can require highly skilled scientific personnel for their operation.

The neuro-behavioral assay described here may fill this gap. Since the frequencies of the EOD and the neural activity of the pacemaker nucleus are identical, and the EOD can be readily quantified, this in vivo behavioral assay provides a robust proxy for examining the effect of anesthetic-drug candidates on neural function in the central nervous system at the systems level. The costs for establishing this assay are roughly one order of magnitude lower than the costs for an electrophysiological setup. The throughput rate of the individual assay is higher than that of traditional electrophysiological testing assays. These advantages make the Neuro-Behavioral Assay an alternative to these traditional physiological-testing methods for initial high-throughput neurophysiological screening of compounds, and for complementing existing in vitro-physiological methods as part of an in-depth evaluation strategy.

### EXAMPLES

### Example 1—The Effect of Urethane and MS-222 Anesthesia on the Electric Organ Discharge of the Weakly Electric Fish Apteronotus leptorhynchus

### Assay

### Animals

For the assay, adult brown ghost knifefish (Apteronotus leptorhynchus; Gymnotiformes, Teleostei), with total lengths of approximately 12-15 cm, are used. The fish are imported from their natural habitat in South and Central America.

At least 1 week before using the assay for the first run, the fish is transferred from a community tank to an isolation tank (50 cm×30 cm×30 cm) equipped with aquarium thermostat heaters and air-driven corner filters. The fish is kept in aquarium water prepared by adding a mixture of inorganic salts (81 mmol/L MgSO₄·7H₂O; 107 mmol/L KCl; 12 mmol/L NaH₂PO₄·2H₂O; 732 mmol/L CaSO₄·2H₂O) to deionized water until a water conductivity of approximately 150-300 μS/cm is reached. Water temperature, determined during the experiments every 10 min with a calibrated high-precision (accuracy+0.05° C.) digital thermometer, is kept in the range of 25.0-28.0° C. across all assays. Variation is limited to 1.0° C. in an individual assay. A 12:12 h light:dark photoperiod is maintained with a timer. The fish are fed red mosquito larvae daily, after the assay run on that day has been completed.

In each isolation tank, an opaque cylindrical plastic tube (length: 190 mm; inner diameter: 38 mm; outer diameter: 42 mm) provides shelter and is readily accepted by the fish, particularly during the light phase, when individuals of this nocturnal species spend most of the time in shelter places. A pair of stainless-steel electrodes is mounted on the inside of each tube (see section 4.1.2, ‘EOD recording,’ below).

Conduction of the assay requires approval by the Institutional Animal Care and Use Committee.

### EOD Recording

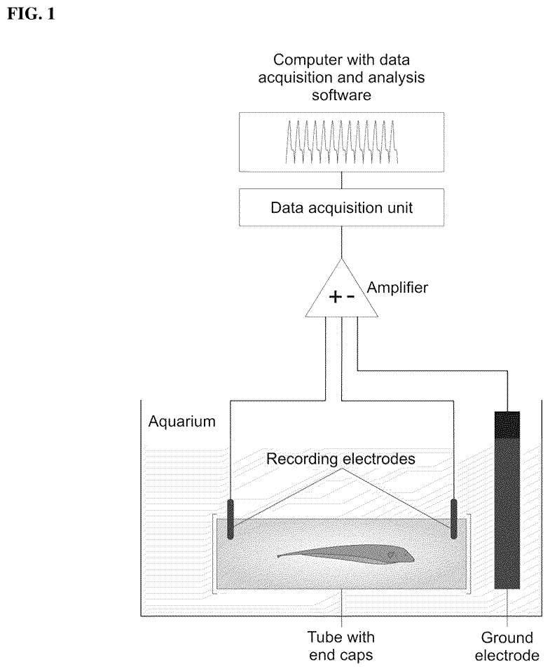

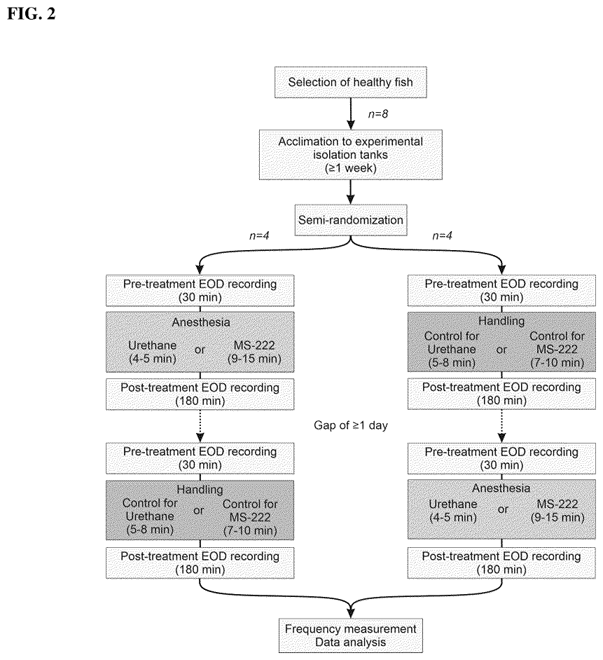

Differential recording of the fish's EOD is done through the pair of stainless-steel electrodes built into the shelter tube (FIG. 1). During the assay, the two open ends of the shelter tube are closed with a coarse plastic mesh netting to ensure that the fish does not leave the tube. This allows the fish's EOD to be recorded continuously without interruptions. At the same time, mesh size is chosen such that sustained water flow through the tube is ensured. A strip of stainless steel is placed in the tank to serve as a ground electrode.

The signal is AC amplified (gain: 30×; low-pass filter: none; high-pass filter: 200 Hz) and then digitized at a sampling rate of 50 kHz.

### Automatic Measurement of EOD Frequency and Rate of EOD Modulations

### Algorithm for Frequency Calculation

The sampled time-voltage data (tᵢ, vᵢ), i=1, . . . , N, are filtered in subsequent, 1-s-long windows using a bandpass filter with frequency band [0.8, 1.2]×f₀, where f₀ is the fundamental frequency determined from the power spectrum of the signal within the associated time window.

The filtered time series data (tᵢ, Vᵢ), i=1, . . . , N, are further processed to determine the EOD frequency at a higher time resolution. First, tuple j containing the indices of all instants where the filtered signal changes sign to a positive value is determined:

j=(i|sgn(Vᵢ₊₁)−sgn(Vᵢ)>0, i=1, . . . ,N)

Then, tuple t⁺ of time instances when the signal crosses the time axis upwards is calculated using interpolation:

$${t}^{+} = ( {t}_{j ( k )} - \frac{{V}_{j ( k )}}{{V}_{j ( k ) + 1} - {V}_{j ( k )}} ( {t}_{j ( k ) + 1} - {t}_{j ( k )} ) \vert k = 1 , … , M )$$

Here M=|j| denotes the number of elements in j (the number of all crossings). Finally, time-frequency pairs (Tₖ, fₖ) are computed as

$${T}_{k} = \frac{{t}^{+} ( k + m ) + {t}^{+} ( k )}{2} , {f}_{k} = \frac{m}{{t}^{+} ( k + m ) - {t}^{+} ( k )}$$

for k=1, . . . , M−m. Here m=10 is the number of averaged time periods used for EOD frequency calculation.

### Temperature Adjustment of EOD Frequency

Since the EOD frequency of A. leptorhynchus is temperature dependent, all measured frequencies are adjusted to a reference temperature (arbitrarily chosen to be 26° C.) so that discharge frequencies at different water temperatures can be compared. This adjustment of a frequency f₁ (in Hz) measured at a temperature T₁ (in ° C.) to the reference temperature T₂=26° C. is calculated as

$${f}_{2} = {f}_{1} {Q}_{10}^{\frac{{T}_{2} - {T}_{1}}{10}}$$

where f₂ is the expected frequency at T₂ and Q₁₀=1.56, as determined empirically.

### Coefficient of Variation

To characterize the variability of the EOD frequency, frequency measurements of the recorded EOD are sampled every 1 min over 30 min, and the coefficient of variation (cv), defined as

cv=(standard deviation/mean)×100(%)

is computed.

### Detection of EOD Modulations

Previous research has shown that transient modulations of the EOD may occur, both spontaneously and through stimulation with electric signal mimicking the EOD of a conspecific. These modulations are brief (several tens of milliseconds) and involve frequency increases. They are commonly referred to as ‘chirps.’

To identify chirp instances, the time-frequency data {(Tₖ, fₖ)}_{k=q}^{q+n}^{_{wind}}, q=1, . . . , M−n_{wind}−1, was considered in a moving time window containing n_{wind}+1 samples. The first and last n_{med} samples in each window are used to establish the instantaneous base frequency before chirping:

f_{base,q}=median({f_{q+p-1}}ₚ₌₁ⁿ^{_{med}},{f_{q+n}_{_{wind}}₋ₚ₊₁}ₚ₌₁ⁿ^{_{med}})

Then the frequency data was shifted and normalized inside each q=1, 2, . . . time window as

$${ϕ}_{p , q} = \frac{{f}_{q + p} - {f}_{base , q}}{{max}_{0 \leq p \leq {n}_{wind}} ( {f}_{q + p} )} , p = 0 , … , {n}_{wind}$$

Inside each q=1, 2, . . . time window, the time axis was also shifted to the midpoint of the window:

$${ξ}_{p , q} = {T}_{q , p} - \frac{{T}_{q} + {T}_{q + {n}_{wind}}}{2} , p = 0 , … , {n}_{wind}$$

We, then, fit the single-parameter function (with parameter α)

$$Φ ( ξ ; α ) = \frac{2 {e}^{αξ}}{1 + {e}^{2 αξ}}$$

to the data set {(ξ_{p,q}, ϕ_{p,q})}ₚ₌₀ⁿ^{_{wind}} for each q=1, 2, . . . time window. This curve fitting was carried out over a fixed search range α∈[αₘᵢₙ, αₘₐₓ] discretized evenly using n_{α}+1 number points. For each q=1, 2, . . . time window, the coefficient of determination R_{q,i}² was computed for each point αᵢ=αₘᵢₙ+i (αₘₐₓ−αₘᵢₙ)/n_{α}, i=0, . . . , n_{α} of the discretized search range. Then, the fitted curve for each q=1, 2, . . . time window as Φ (ξ; [symbol] ), with

$${\hat{i}}_{q} = {argmax}_{i} ( {R}_{q , i}^{2} )$$

is reported, and collected into a tuple q_{ε} the instances of time windows where the coefficient of determination

$$= {max}_{i} ( {R}_{q , i}^{2} )$$

of the fitted curve and the maximum frequency rise

$$Δ {f}_{q} = {max}_{0 \leq p \leq {n}_{wind}} ( {f}_{q + p} - {f}_{base , q} )$$

are both above thresholds ε_{R}_{²} and ε_{f}, respectively:

q_{ε}=(q| [symbol] >ε_{R}_{²},Δf_{q}>ε_{f})

Subsequently, using Algorithm 1, contiguous segments of successive windows are identified within q_{ε} and for each segment the time window q associated with the maximum [symbol] value is reported together with the corresponding parameter [symbol] and maximum frequency rise Δf_{q}.

Algorithm 1: Identification of contiguous segments of successive
time windows within q_{ε}

```
Input: [symbol] , Δf_{q}, q = 1, ... , M − n_{wind} − 1
Output: W, R, A, F arrays containing for each contiguous segment the
index associated with maximum [symbol]  value and associated parameters
I₁ ← [ ], I₂ ← [ ] arrays containing first and last indices of contiguous
segments
for q = 1, ... , M − n_{wind} − 2 do
  if ( [symbol]   ≤ ε_{R}_{²} or Δf_{q} ≤ ε_{f}) and [symbol]  > ε_{R}_{²} and Δf_{q+1} > ε_{f}
    I₁ ← [I₁, q]
  elseif [symbol]    > ε_{R}_{²} and Δf_{q} > ε_{f} and ( [symbol]   ≤ ε_{R}_{²} or Δf_{q+1} ≤ ε_{f})
    I₂ < [I₂, q]
  end
end
if I₁(1) > I₂(1)
  I₁ ← [1, I₁]
end
if I₁ (end) > I₂(end)
  I₂ ← [I₂, M − n_{wind} − 1]
end
Let arrays W, R, A, F be of size Length(I₁)
for k = 1, ... , Length(I₁) do
  $w ← {argmax}_{{I}_{1} ( k ) < q \leq {I}_{2} ( k )} ( \hat{{R}_{2}^{q}} )$
  W(k), R(k), A(k), F(k) ← w, [symbol] , [symbol] , Δf_{w}
end
```

Nonlinear regression is performed on data points within these time windows using the three-parameter model Ψ(ξ; α, Δf, τ)=Δf Φ(ξ−τ; α) with initial parameters α= [symbol] , Δf=Δf_{q}, and τ=0. Finally,

$${\hat{T}}_{chirp , q} = \frac{{T}_{q} + {T}_{q + {n}_{wind}}}{2} + {\hat{τ}}_{q}$$

as time instance, [symbol] as maximum frequency rise and \hat{d}_{chirp,q}=ln(7+4\sqrt{3})/\hat{α}_{q} is reported as duration of a chirp for each q reported time window with parameter estimates \hat{α}_{q}, [symbol] , \hat{τ}_{q}. Here the formula for chirp duration corresponds to the half prominence of Ψ(ξ; \hat{α}_{q}, [symbol] , \hat{τ}_{q}).

The implemented values of parameters for the above-described method are summarized in Table 1.

TABLE 1
Parameters chosen for the chirp identification
method presented in section 4.2.4
‘Detection of EOD modulations.’
Parameters for chirp identification

| Parameter (unit) | Value |
|---|---|
| n_{wind} (1) | 80 |
| n_{med} (1) | 15 |
| ε_{R}_{²} (1) | 0.45 |
| ε_{f} (Hz) | 8 |
| n_{α} (1) | 200 |
| αₘᵢₙ (1/s) | 100 |
| αₘₐₓ (1/s) | 800 |

### Demonstration of the Applicability of the Assay

To demonstrate the applicability of the assay for physiological evaluation of water-soluble anesthetics, the effects of urethane and MS-222 (also known as tricaine methanesulfonate or 3-aminobenzoic acid ethyl ester methanesulfonate) on the EOD frequency and chirp rate were examined in two experiments.

### Study Design

### Experiment 1

In Experiment 1, whether urethane or MS-222 anesthesia had an effect on EOD frequency was examined. The experimental design is outlined in FIG. 2. To determine the baseline EOD frequency, the fish's EOD was recorded for 30 min. Then, the fish was transferred into a glass beaker containing either 2.5% urethane (Acros Organics, New Jersey, USA) or 0.02% MS-222 (Western Chemical, Washington, USA), each dissolved in water from the fish's isolation tank. As a control, the fish was moved into an identical glass beaker containing aquarium water only. The temperatures of the anesthetic solutions or the aquarium water in the beaker were similar (±1° C.) to the temperature of the water in the fish's isolation tank.

The fish was left in the anesthetic solution until both anal fin undulation and opercular movement ceased. It was then returned to its isolation tank where recording of the EOD continued for 180 min. For urethane anesthesia, treatment including transfer to and from the beaker took between 4-5 min. MS-222 anesthesia required longer exposure to the anesthetic so that the entire treatment lasted between 9-15 min. Control experiments were designed such that the handling of the fish and the time the fish spent outside its isolation tank were similar to the corresponding anesthesia experiments (urethane control: 5-8 min; MS-222 control: 7-10 min).

The effect of urethane and MS-222 was tested in 8 fish each. Every anesthesia experiment was paired with a control experiment, and they were run on 2 different days. The order of the anesthesia experiment and the control experiment was determined using a semi-random design, ensuring that in 4 fish the urethane-anesthesia experiment was followed by the control experiment, whereas in the other 4 individuals the control was carried out before the urethane experiment. Apart from this constraint, the assignment of the 8 experimental pairs to the fish was random.

Out of the 12 fish in Experiment 1, 4 fish were used in both the urethane and MS-222 experiments (and corresponding control experiments), whereas the remaining 8 fish were involved in the testing of only one of the two anesthetics. Any fish that was reused was given ≥1 week between urethane anesthesia and MS-222 anesthesia to ensure that the fish was well-adjusted before further testing. In no case was there any indication that prior exposure to one of the two anesthetics had an impact on the effect of the other anesthetic, as determined based on EOD frequency.

### Experiment 2

After Experiment 1 established that both urethane and MS-222 affect the EOD, Experiment 2 was used to examine the time course of the changes in EOD frequency during the fish's exposure to the respective anesthetic.

First, the fish's baseline EOD was recorded for 10 min. Then, the entire electrode tube with the fish inside was transferred from the isolation tank into a plastic tank (30 cm×20 cm×20 cm) containing either 2.5% urethane or 0.02% MS-222 in aquarium water. Based on the average time the fish had spent in the anesthetic solution in Experiment 1, the fish's EOD was recorded in the plastic tank for about 6 min if urethane was used as an anesthetic, and about 10 min if MS-222 was employed.

### Statistical Analysis

The Related-Samples Sign Test was used to assess differences in temperature-adjusted EOD frequencies between groups. Significance levels were set at p<0.01 (2-tailed).

### Results

### Baseline EOD Frequency

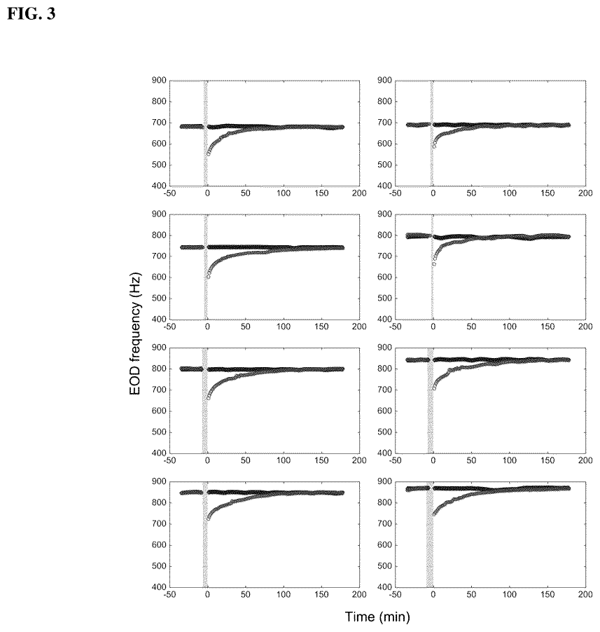

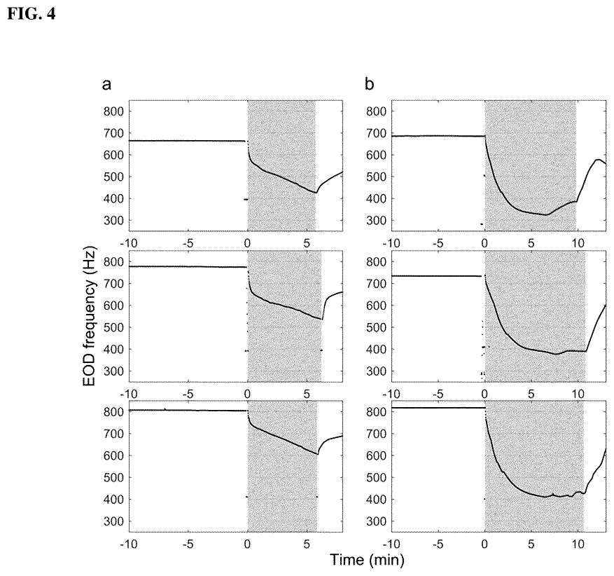

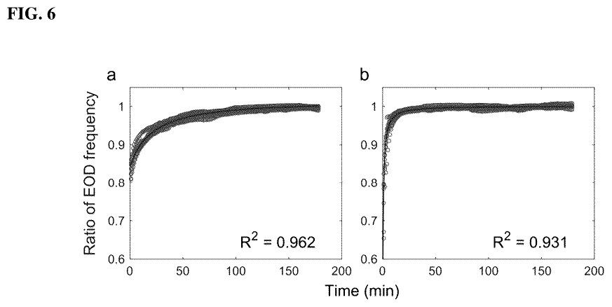

The median frequencies of the EODs generated by the 12 fish in this study ranged from 626 Hz to 869 Hz (29 measurements taken at equal intervals during the 32 pre-treatment recordings) (FIGS. 3, 5). While these frequencies differed between fish, they were highly stable in each individual during the 30-min pre-treatment recordings, regardless of whether these baseline recordings were followed by handling (cv: 0.01-0.20; mean: 0.11; median: 0.10; n=12 fish, with 2 experiments and 29 frequency measurements each), or urethane or MS-222 anesthesia (cv: 0.04-0.44; mean: 0.19; median: 0.18; n=12 fish, with 2 experiments and 29 frequency measurements each).

### Effect of Anesthesia on EOD Frequency

### Urethane

In each of the 8 fish, urethane anesthesia resulted in a fast and pronounced drop in EOD frequency. Immediately after returning the anesthetized fish from the beaker with the urethane solution to their isolation tanks, the EOD frequency was 104-142 Hz (median: 133 Hz) lower than the corresponding median baseline frequency (FIG. 3, red circles). This decrease in frequency sustained for up to 3 hours (for details, see section 4.3.3.3 ‘Recovery of EOD frequency after urethane and MS-222 anesthesia,’ below). Comparison of the medians of the EOD frequencies during the 30 min before and after the urethane anesthesia revealed a significant difference, ranging from −42 Hz to −75 Hz (p=0.008, Related-Samples Sign Test, n=8 fish). In the corresponding control experiments, no significant effect was observed when comparing the medians of the EOD frequencies during the 30 min preceding the handling and during the 30 min immediately following the handling (p=1.000, Related-Samples Sign Test; n=8 fish) (FIG. 3, blue diamonds).

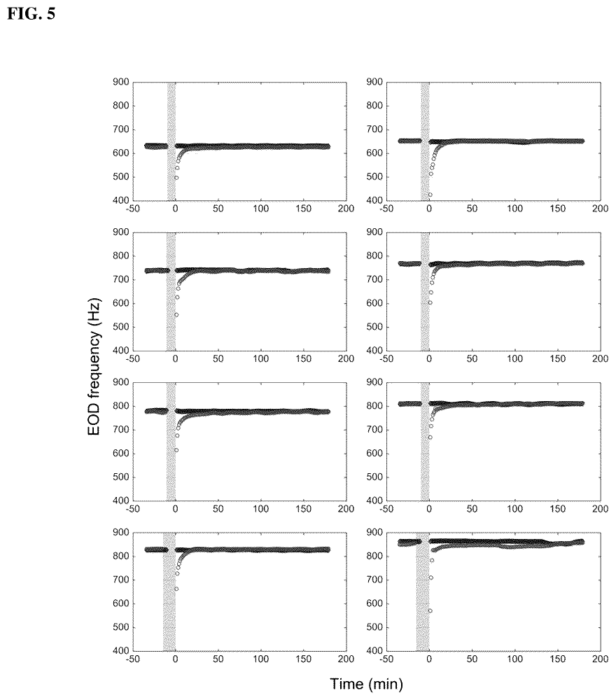

Analysis of the temporal dynamics of the EOD frequency during urethane anesthesia revealed two phases (FIG. 4a): A first phase lasting for roughly 30 s, during which the frequency dropped rapidly by approximately 100 Hz; and a second phase spanning the remaining time of the fish's exposure to the anesthetic (about 5 min), which was characterized by a continued, though slower, linear decrease of frequency with time. During the first phase, the difference between the instantaneous frequency and the line regressed on the second phase (not shown) was inversely proportional to time.

### MS-222

In each of the 8 fish, MS-222 anesthesia resulted in a fast drop in EOD frequency, which was more pronounced than the decrease observed after urethane anesthesia. Immediately after returning the anesthetized fish from the beaker with the MS-222 solution to their isolation tanks, the EOD frequency was 129-279 Hz (median: 168 Hz) lower than the corresponding median baseline frequency (FIG. 5, red circles). Recovery of the baseline EOD frequency was faster than after urethane anesthesia (for details, see section 4.3.3.3 ‘Recovery of EOD frequency after urethane and MS-222 anesthesia’, below). Comparison of the medians of the EOD frequencies during the 30 min before and after the MS-222 anesthesia revealed a significant difference, ranging from −8 Hz to −18 Hz (p=0.008, Related-Samples Sign Test; n=8 fish). In the corresponding control experiments, no significant effect was observed when comparing the medians of the EOD frequencies during the 30 min preceding the handling and during the 30 min immediately following the handling (p=0.727, Related-Samples Sign Test; n=8 fish) (FIG. 5, blue diamonds).

The time course of frequency change during MS-222 anesthesia differed markedly from that during urethane anesthesia (FIG. 4b). The first 7-8 min of the fish's exposure to the anesthetic demonstrated a rapid decrease roughly proportional to the inverse square of time. During the remaining 3-4 min of anesthesia, the EOD frequency fluctuated around a plateau near the lowest frequency (observed in 2 fish) or even showed signs of a return towards baseline levels (found in 1 fish).

### Recovery of EOD Frequency After Urethane and MS-222 Anesthesia

After both urethane and MS-222 anesthesia, the EOD frequency started to return to baseline levels as soon as the fish was transferred from the beaker with the anesthetic solution to its isolation tank (FIGS. 3, 5). The time course of recovery, expressed as the ratio of temperature-adjusted EOD frequency vs. baseline frequency, across all measured fish can be described by the following function:

$$F ( t ) = a + \frac{( c - a ) t + d {t}^{2}}{b + t} , 0 \leq t \leq {t}_{r} \qquad ( 1 )$$

Here t=0 corresponds to the time instance when the fish was returned to its tank from the beaker with the anesthetic solution. At this time instance, the EOD frequency is given by parameter a. As t increases, F(t) approaches the line l(t):=c+d t, while parameter b controls the shape of the curve between a and l(t). It is important to note that this model is valid only until the frequency recovers to the baseline at time tᵣ. This time instance is slightly different for each fish and varies around 3 hours for urethane and 1 hour for MS-222.

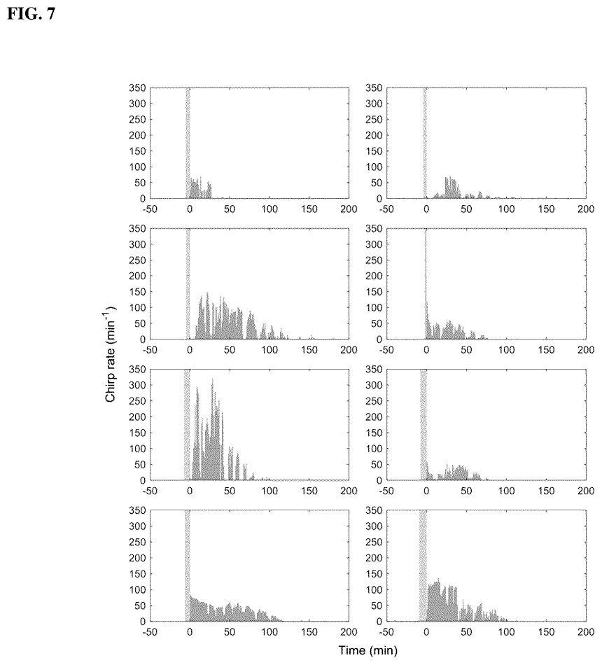

Parameters a, b, c, and d were identified using robust nonlinear regression. The resulting curves are displayed together with ratios based on the measured data points in FIG. 6. The parameters of the fitted model, together with their 95% confidence intervals, are given in Table 2.

TABLE 2
Parameters identified for the model described by Equation 1. Parameter identification was
performed by robust nonlinear regression on the ratio of temperature adjusted EOD frequency
vs. baseline frequency data points associated with the recovery part of urethane or MS-222
anesthesia. For detailed information see the Brief Descriptions of FIGS. 3, 5.
Model Parameter Estimates

| Parameter | Urethane | | MS-222 | |
|---|---|---|---|---|
| (unit) | Estimate | Confidence interval (95%) | Estimate | Confidence interval (95%) |
| a (1) | 0.84285 | [0.84003, 0.84568] | 0.35844 | [0.31041, 0.40648] |
| b (min) | 21.333 | [19.582, 23.084] | 0.51999 | [0.46665, 0.57332] |
| c (1) | 1.0228 | [1.0181, 1.0275] | 1.0024 | [1.0017, 1.0031] |
| d (min⁻¹) | −2.8717 × 10⁻⁵ | [−5.2089, −0.53453] × 10⁻⁵ | −9.0311 × 10⁻⁶ | [−14.581, −3.4816] × 10⁻⁶ |

### Effect of Anesthesia on Chirp Rate

### Urethane

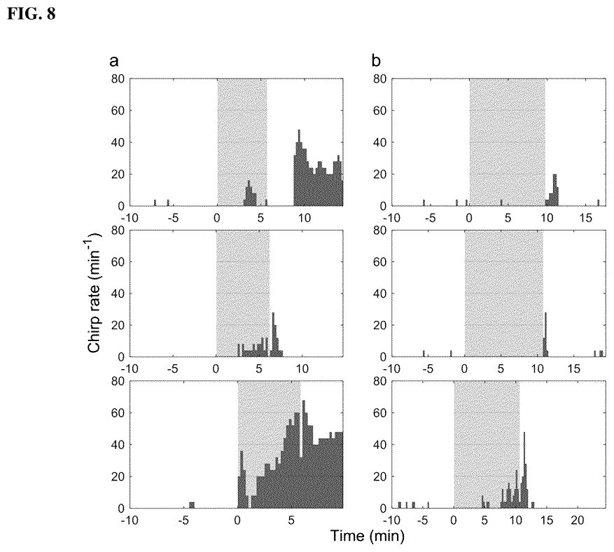

In each of the 8 fish, urethane anesthesia resulted in a manifold increase in the number of chirps produced, compared to baseline levels (FIG. 7). During the 30 min immediately following the anesthesia, the number of chirps varied among individual fish between 568-4434 chirps (mean: 1852 chirps; median: 1541 chirps; n=8 fish with 1 experiment each) and was significantly higher than the number of chirps generated during the 30-min-pre-treatment recording (p=0.008; Related-Samples Sign Test; n=8 fish). In the corresponding control experiments, no significant difference was found between the number of chirps produced during the 30 min before and the 30 min after handling (p=0.219; Related-Samples Sign Test; n=8 fish).

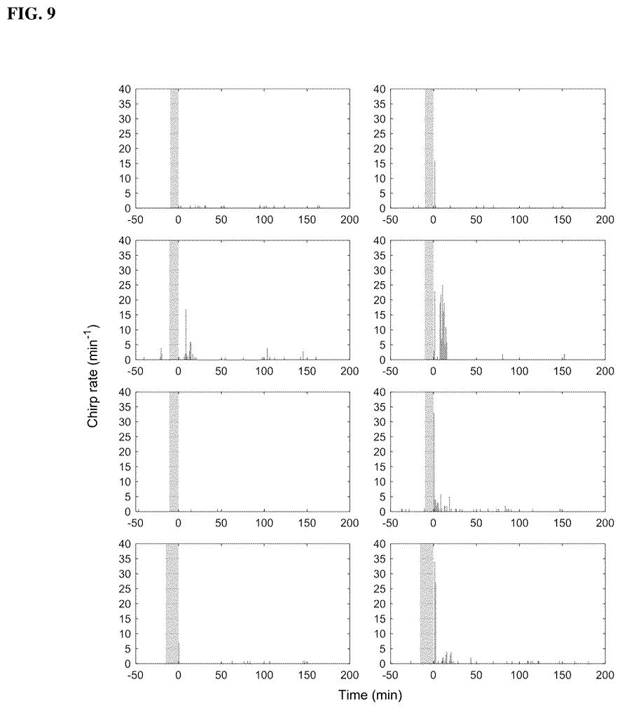

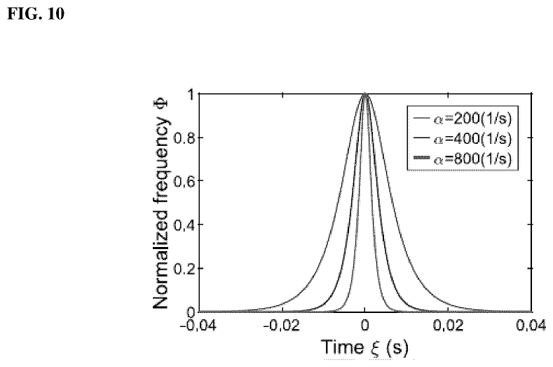

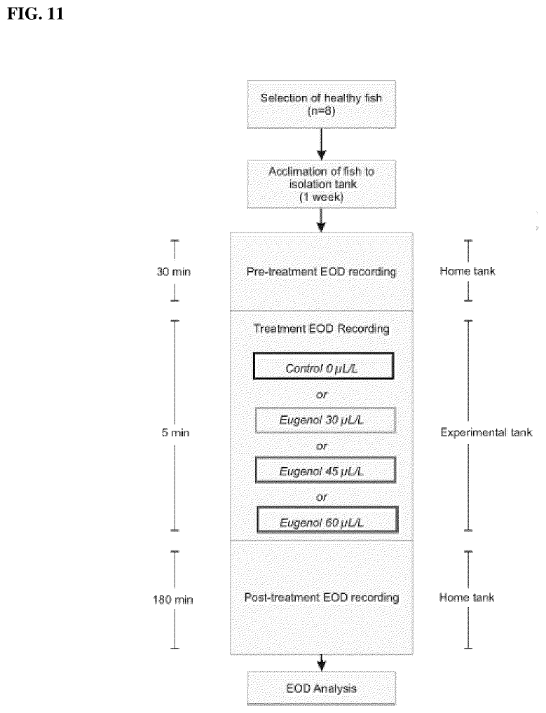

During urethane anesthesia, the temporal dynamics of chirping were highly variable among the 3 individual fish examined (FIG. 8a). In the first individual, a surge of chirps occurred about 3 min after the fish's transfer into the anesthetic solution, lasting for approximately 1 min. In the second fish, a surge of chirps emerged at about the same time into the anesthesia but continued throughout, and beyond, the anesthesia. In the third individual, a surge of chirping developed as soon as the fish's exposure to the anesthetic started and continued after the fish was returned to its isolation tank with aquarium water.

### MS-222

MS-222 anesthesia resulted in a rise in the rate of chirping (FIG. 9), but this increase was less pronounced than the increase after urethane anesthesia (cf. FIG. 7). During the 30 min immediately following MS-222 anesthesia, the number of chirps varied among individual fish between 1-156 chirps (mean: 48 chirps; median: 30 chirps; n=8 fish with 1 experiment each) and was significantly higher than the number of chirps produced spontaneously during the 30-min-pre-treatment period (p=0.008; Related-Samples Sign Test; n=8 fish). Chirp rates remained elevated, although at rather low levels, up to approximately 3 hours after anesthesia. No significant difference was observed in the number of chirps between the 30 min preceding handling and the 30 min following handling in the corresponding control experiments (p=0.031; Related-Samples Sign Test; n=8 fish).

Like the temporal dynamics of chirping during urethane anesthesia, the time course of chirping during MS-222 anesthesia was highly variable among individuals (FIG. 8b). In the first fish, no increase in chirping, compared to baseline levels, was observed. Contrarily, chirps were entirely absent in the second fish. In the third fish, the rate of chirping gradually increased until the end of anesthesia.

### REFERENCES CITED

Carmody J J (2009) Some scientific reflections on possible mechanisms of general anaesthesia. Anaesth Intensive Care 37:175-189. https://www.ncbi.nlm.nih.gov/pubmed/19400482
Chitilian H V, Eckenhoff R G, Raines D E (2013) Anesthetic drug development: novel drugs and new approaches. Surg Neurol Int 4:S2-S10. https://www.ncbi.nlm.nih.gov/pubmed/23653886
Enger P S, Szabo T (1968) Effect of temperature on the discharge rates of the electric organ of some gymnotids. Comp Biochem Physiol 27:625-627. https://www.ncbi.nlm.nih.gov/pubmed/5758391
Engler G, Fogarty C M, Banks J R, Zupanc G K H (2000) Spontaneous modulations of the electric organ discharge in the weakly electric fish, Apteronotus leptorhynchus: a biophysical and behavioral analysis. J Comp Physiol A 186:645-660. https://doi.org/10.1007/s003590000118
Engler G, Zupanc G K H (2001) Differential production of chirping behavior evoked by electrical stimulation of the weakly electric fish, Apteronotus leptorhynchus. J Comp Physiol A 187:747-756. https://doi.org/10.1007/s00359-001-0248-8
Lca W A, Xi J, Jadhav A, Lu L, Austin C P, Simconov A, Eckenhoff R G (2009) A high-throughput approach for identification of novel general anesthetics. PLOS One 4:07150. https://www.ncbi.nlm.nih.gov/pubmed/19777064
Mckinstry-Wu A R, Bu W, Rai G, Lea W A, Weiser B P, Liang D F, Simconov A, Jadhav A, Maloney D J, Eckenhoff R G (2015) Discovery of a novel general anesthetic chemotype using high-throughput screening. Anesthesiology 122:325-333. https://www.ncbi.nlm.nih.gov/pubmed/25603205
Rivera R, Antognini J F (2009) Perioperative drug therapy in elderly patients. Anesthesiology 110:1176-1181. https://www.ncbi.nlm.nih.gov/pubmed/19352149
Sîrbulescu R F, Ilieş I, Zupanc G K H (2009) Structural and functional regeneration after spinal cord injury in the weakly electric telcost fish, Apteronotus leptorhynchus. J Comp Physiol A 195:699-714. http://www.ncbi.nlm.nih.gov/entrez/query.fcgi?cmd=Retrieve&db=PubMed&dopt=Citati on&list_uids=19430939
Welliver M, Rugari S M (2009) New drug, fospropofol disodium: a propofol prodrug. AANA J 77:301-308. https://www.ncbi.nlm.nih.gov/pubmed/19731849
Woll K A, Eckenhoff R G (2018) High-throughput screening to identify anesthetic ligands using Xenopus laevis tadpoles. Methods Enzymol 602:177-187. https://www.ncbi.nlm.nih.gov/pubmed/29588028
Zupanc G K H, Banks J R, Engler G, Beason R C (2003) Temperature dependence of the electric organ discharge in weakly electric fish. In: Ploger B J, Yasukawa K (eds) Exploring animal behavior in laboratory and field: an hypothesis-testing approach to the development, causation, function, and evolution of animal behavior. Academic Press, Amsterdam, pp 85-94
Zupanc G K H, Sîrbulescu R F, Nichols A, Ilies I (2006) Electric interactions through chirping behavior in the weakly electric fish, Apteronotus leptorhynchus. J Comp Physiol A 192:159-173. https://doi.org/10.1007/s00359-005-0058-5
Zupanc M M, Engler G, Midson A, Oxberry H, Hurst L A, Symon M R, Zupanc G K H (2001) Light-dark-controlled changes in modulations of the electric organ discharge in the telcost Apteronotus leptorhynchus. Anim Behav 62:1119-1128. https://www.sciencedirect.com/science/article/pii/S0003347201918676

### Example 2—The Effect of Eugenol Anesthesia on the Electric Organ Discharge of the Weakly Electric Fish Apteronotus leptorhynchus

Over the last few decades, legal requirements for minimizing pain, suffering, or distress inflicted upon protected animals during experiments have prompted the search for suitable anesthetics and the optimization of methods used for anesthesia. Ideal anesthetics are cheap and readily available; easy to administer and have minimal toxic effects on the animal, the handler, and the environment; provide a wide margin of safety; induce anesthesia rapidly and lead to quick recovery after removal of the animal from the anesthetic. Moreover, if the research project involves examination of nervous function, the effect of the anesthetic on the neural system under study should be minimal.

For anesthesia in fish, no currently available drug meets all the above requirements, but several agents satisfy some of them. For each of the common drugs, doses have been established that induce and/or maintain anesthesia in selected fish taxa. However, a significant gap in knowledge exists about possible neural effects of these anesthetics. To address this issue, a neuro-behavioral assay was recently developed. The unique feature of this in-vivo tool is that a well-defined and readily accessible behavior serves as a proxy of the pattern of the neural command signals that drive the frequency of this behavior.

The behavior utilized by the assay is the electric organ discharge (EOD) of the weakly electric fish Apteronotus leptorhynchus. These discharges are generated continuously at frequencies of approximately 650-1,000 Hz, with males occupying a frequency band between 700 Hz and 1,000 Hz, and females discharging within a frequency band ranging from 650 Hz to 800 Hz, at a water temperature of 26° C. The EOD is generated by an electric organ composed of axonal terminals (so-called electrocytes) of modified spinal motoneurons (so-called electromotoneurons). The synchronized discharges of individual electrocytes constitute the EOD.

The frequency of the EOD is controlled by the frequency of the oscillations of a central pattern generator in the medulla oblongata, the pacemaker nucleus (Pn). Its neural output drives the EOD in a one-to-one fashion, i.c., the frequency of the EOD equals the frequency of the synchronized oscillations of the Pn. Thus, the frequency of the EOD can be used as a proxy of the frequency of the Pn oscillations.

In fish similar in size to the ones used in the present study, the Pn consists of roughly 200,000 cells, of which approximately 5,000 are neurons. However, neuroanatomical and neurophysiological evidence, as well as simulations based on computational modeling, suggest that a relatively small neural network of approximately 90 pacemaker cells and 20 relay cells is sufficient to produce the sustained high-frequency oscillations of the Pn. The pacemaker cells are connected via chemical and electrotonic synapses with each other and the relay cells. The latter project to the spinal electromotoneurons.

Modulations of the EOD involve changes in frequency and/or amplitude. Some of them are limited to a few milliseconds. The most common type of these transient modulations is chirps, which consist of specific frequency increases and amplitude decreases. At the other extreme are modulations that become manifest in the course of weeks, such as frequency decreases experimentally induced by β-estradiol; they mimic the development of the sexual dimorphism in EOD frequency in A. leptorhynchus.

Neurophysiological experiments and simulations based on computational modeling have suggested two major types of neural mechanisms underlying EOD modulations. One type operates within the Pn. For example, changes in potassium equilibrium potential caused by alterations in the capacity of astrocytes to buffer extracellular K⁺ have been proposed to mediate changes in EOD frequency. A second type is based on input received by the Pn from other brain regions. One of these inputs is potentially involved in mediating the effect of eugenol on the EOD, as will be discussed in detail below. This excitatory input is received by the relay cells from the mesencephalic sublemniscal prepacemaker nucleus (SPPn) via NMDA receptors. The SPPn, in turn, is under tonic inhibition from a subnucleus of the nucleus electrosensorius in the diencephalon, the nE↓. The latter input utilizes GABA as a transmitter and activates GABA_{A} receptors.

A neuro-behavioral assay is employed to examine the effect of eugenol on the EOD of A. leptorhynchus. This phenylpropanoid is the major active component of clove oil; other active ingredients are isoeugenol and methyl-eugenol. Clove oil and each of its three active ingredients have been widely used as fish anesthetics. Yet, only very recently possible effects of eugenol on physiological functions in the central nervous system (CNS) have attracted attention by investigators. The results of our investigation indicate a pronounced, dose-dependent effect of eugenol on the frequency of the neural oscillations of the Pn, commencing within less than a minute after the fish's exposure to the anesthetic and lasting for up to 30 min after the fish's removal from the immersion solution. These findings call for caution when conducting neurophysiological experiments on fish under general anesthesia with eugenol.

### Materials and Methods

### Animals

A total of 8 adult (approximately 2-year-old) A. leptorhynchus, collected in their natural habitat in Colombia and obtained through a tropical fish importer (Segrest Farms, Gibsonton, Florida, USA), were used in this study. Their total lengths (body weights) ranged from 122 mm to 157 mm (4.0 g to 6.9 g), with medians of 135 mm and 6.1 g, respectively. The baseline EOD frequencies of the fish indicated that this sample contained both males (high frequencies) and females (low frequencies).

The fish were kept isolated in their home tanks (50 cm×30 cm×30 cm) under a 12:12-h light:dark photoperiod at water temperatures of 26-29° C., conductivities of 180-310 μS/cm, and pH values of 7.7-7.9. Each tank was equipped with an opaque cylindrical tube (length: 190 mm; inner diameter: 38 mm; outer diameter: 42 mm), which provided shelter for the fish. During the experiments, the water temperature was sampled every 10 min with a calibrated digital thermometer (Fisher Brand, Model 15-077-8, 11.705,843; Thermo Fisher Scientific, Waltham, Massachusetts, USA; accuracy±0.05° C.).

### EOD Recording

Differential recording of the fish's EOD was performed through a pair of stainless-steel electrodes built into the shelter tube (FIG. 1). During the experiment, the open ends of the tube were closed with a course plastic mesh netting to ensure that the fish remained close to the recording electrodes, thereby yielding high-quality recordings. This restraint is minimally invasive, as, particularly during daytime, the fish remain in shelter tubes with open ends for prolonged periods of time, often for several hours. The recorded EOD signal was amplified and digitized following a protocol established previously.

### Calculation of EOD Frequency

For calculation of EOD frequency, the method described by Eske et al. (2023) was implemented using MATLAB version R2021b with the following modifications: The time series data were filtered in a moving time window using a bandpass filter with frequency band [0.4, 1.4]×f_{d,k}, where f_{d,k} is the dominant frequency of window k inside the frequency range [0.9, 1.1]×f_{d,k-1} determined by the previous window's dominant frequency f_{d,k-1}. For each window k=1, 2, . . . , the dominant frequency f_{d,k} was computed based on the power spectrum of the time series data using fast Fourier transform and the “findpeaks” function of MATLAB. The initial dominant frequency f_{d,0} was determined as the dominant frequency of the baseline recording.

### Temperature Adjustment

All frequencies were adjusted to a reference temperature of 26° C., using a Q₁₀ of 1.56.

### Coefficient of Variation

The variability of sampled EOD frequency measurements was characterized by computing the coefficient of variation (cv), defined as

$$cv = \frac{standard deviation}{mean} 100 ( \% ) . \qquad ( 1 )$$

### Chirp Detection

The method described in Eske et al. (2023) was employed for the detection of chirps. This method assumes that the frequency shape of chirps normalized by the peak frequency rise is described in time by function

$$Φ ( ξ ; α ) = \frac{2 {e}^{αξ}}{1 + {e}^{2 αξ}} \qquad ( 2 )$$

where ξ is the time coordinate measured from the chirp's peak frequency and α is an unknown parameter identified by the chirp detection method (FIG. 2). This function closely matches the shape of type-2 chirps, and, though to a lesser extent, the shape of type-1 chirps. Since the present study investigates the effect of anesthesia on type-2 chirps, only chirps with maximum frequencies of less than 150 Hz were included in our analysis, which is a defining property of type-2 chirps. By contrast, type-1 chirps exhibit maximum frequencies between 200 Hz and 350 Hz.

### Model Fitting of EOD Frequency Recovery

The EOD frequency of each recording was normalized with respect to its baseline frequency. For each eugenol concentration, the following model was fitted on the resulting normalized frequency data points after the fish's exposure to the anesthetic solution:

$$F ( t ) = a + \frac{( c - a ) t + {dt}^{2}}{b + t} \qquad ( 3 )$$

Here t=0 corresponds to the time instant when the fish was returned to its home tank. The fitting of model parameters a, b, c, and d was carried out according to the method described in.

### Statistical Analysis

Statistical analysis of the data was performed using R version 4.2.3. Throughout this paper, significance levels were set at p<0.01 (2-tailed).

To assess differences in the change of EOD frequency and chirp rate after anesthesia, the Related-Samples Sign Test using the “rstatix” package in R was employed. To minimize the number of fish involved in our experiments, we chose the smallest number of fish n required for significance under the expected test statistic (0 or n): n=[1−log₂(0.01)]=8 was chosen.

To assess dose-dependent differences in recovery, Split-Plot ANOVA based on the following linear mixed effects model were used:

Yᵢⱼₖ=δ₀+δ₁cᵢ+βⱼ₍ᵢ₎+δ₂tₖ+δ₁₂cᵢtₖ+εᵢⱼₖ (4)

Here, Yᵢⱼₖ denotes the measured normalized frequency data after anesthesia, with the indices being associated with eugenol treatment i∈{0, . . . ,3} (i=0 corresponds to control), fish j∈{1, . . . ,8}, and time instance k∈{1, . . . ,360}. Parameter δ₀ is the overall mean, δ₁ denotes the effect of eugenol concentration cᵢ, βⱼ₍ᵢ₎˜ [symbol] (0, σ_{β}²) is the random effect of fish j under treatment i, δ₂ is the effect of time tₖ=k/2 (min), δ₁₂ is the concentration-dependent effect of time, and εᵢⱼₖ˜ [symbol] (0, σ_{ε}²) is the measurement error (σ_{β}² and σ_{ε}² are unknown variances).

This model assumes that, for a given treatment, the recovery curve of normalized frequency is independent of the fish. To fit this model on the measured data and to assess significance, the “ImerTest” package in R was used.

To determine the time duration of recovery at each eugenol concentration (i>0), time windows q=0, 1, . . . , 345 Eq. 4 on the normalized frequency data Yᵢⱼₖ associated with the given eugenol concentration and control were fitted, and with the time window k∈{q+1, . . . , q+15}. For each r=1, 2, . . . , the null hypothesis was tested that in time windows q=1, . . . , r, δ₁=0 and δ₁₂=0 both hold. The Bonferroni correction was applied to assess significance and assigned the recovery time tᵣ to the first r value where the null hypothesis was not rejected.

### Experimental Design

To examine the effect of eugenol on EOD frequency and chirping behavior, the following experiments were conducted using 8 fish (FIG. 11): First, the baseline EOD was recorded in the fish's home tank (50 cm×30 cm×30 cm) for 30 min. Then, for the 5-min treatment session, the shelter tube with the fish was transferred to a smaller (experimental) tank (30 cm×20 cm×20 cm) containing either eugenol dissolved in aquarium water at a certain concentration (30 μL/L, 45 μL/L, or 60 μL/L; ‘test condition’) or aquarium water only (‘control condition’). The temperature in the experimental tank was similar to the temperature in the home tank (median difference: ±0.2° C.). Finally, the shelter tube with the fish was returned to the home tank, where recording continued for 180 min. Throughout the 215 min of pre-treatment, treatment, and post-treatment, the recording of the EOD was interrupted only for a few seconds during which the fish was transferred between its home tank and the experimental tank. The order of the four treatment conditions was determined using a randomized complete block design. Each test or control experiment in a given fish was conducted on a different day.

### Results

### Effect of Eugenol Anesthesia on Locomotor Activity

Most of the time, fish in the shelter tube maintained an upright position, with their longitudinal axis parallel to the walls of the tube. Forward and backward swimming occurred frequently and appeared to be mediated primarily by high-frequency undulations of the anal fin. Sporadically, the fish made 180° turns around its vertical axis.

Upon exposure of the fish to eugenol, body movements, including undulation of the anal fin, slowed down. When these movements ceased, the fish lost equilibrium and lay on one side. Quantitative analysis of the locomotor activity in 8 fish revealed that the time after which body movements ceased was dose dependent: 1-3 min (mean: 1.8 min) at 30 μL/L eugenol, 1-2 min (mean: 1.4 min) at 45 μL/L eugenol, and 1 min in each fish at 60 μL/L eugenol.

Whereas the time after which the movement of the body stopped at a given eugenol concentration was rather robust, the time after which the anal fin ceased undulating was quite variable. At a concentration of 30 μL/L, in 3 out of 8 fish, the anal fin continued undulating throughout the 5-min exposure of the fish to eugenol. In the other 5 fish, undulation stopped after 1-3 min. At a concentration of 45 μL/L, in 1 out of 8 fish the anal fin continued to undulate throughout the 5-min eugenol anesthesia. In the other 7 fish, the anal fin ceased undulating after 1-3 min. At a concentration of 60 μL/L, the anal fin stopped undulating in each of the 8 fish during anesthesia; this effect required eugenol exposure times ranging from 1 to 4 min.

### Effect of Eugenol Anesthesia on EOD Frequency

For determining the baseline EOD frequency of each of the 8 fish, the 30-min recordings of their EODs before each treatment (exposure to water from the home tank only or to eugenol at one of the three concentrations) were used. Then, the median frequencies at 30-s intervals within 1-min time windows of each of these 30-min recordings were computed, yielding a total of 59 median frequency values. As expected, based on our selection criterion, the frequencies of individual fish differed substantially (FIG. 4). However, the EOD frequencies within each of the 32 pre-treatment time periods of 30 min were highly stable, as indicated by the small cv values ranging from 0.12% to 0.59%.

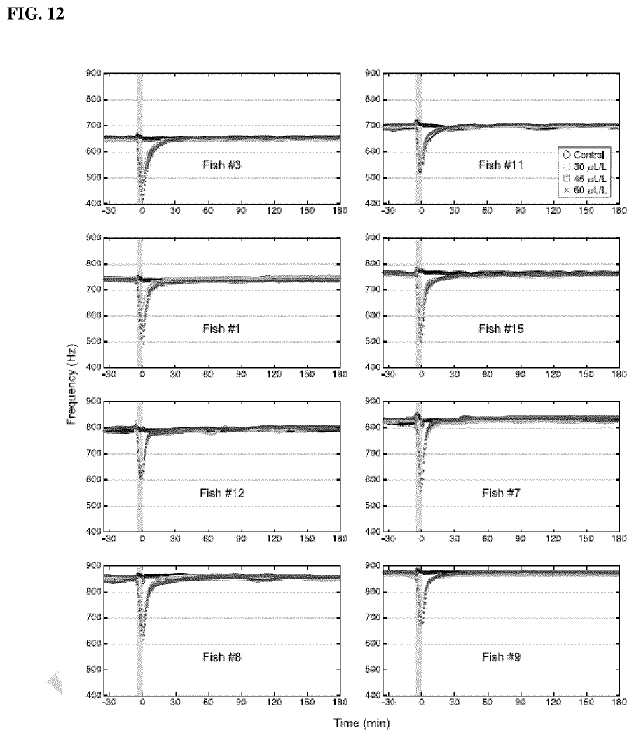

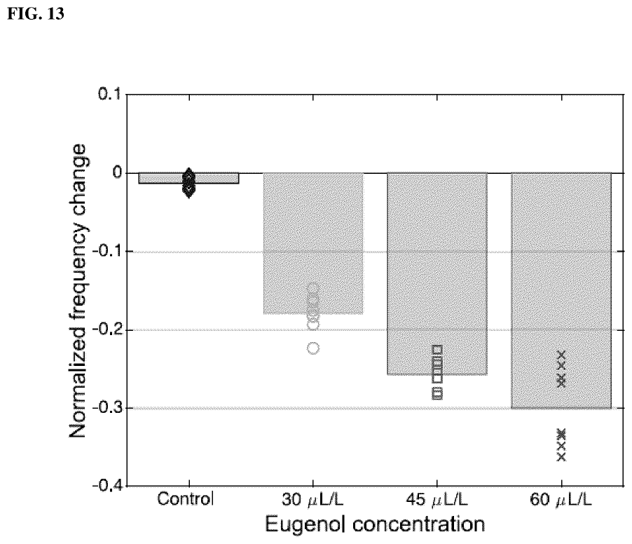

In each of the 8 fish examined, eugenol anesthesia resulted, within minutes, in a pronounced decrease in EOD frequency (FIG. 12). The magnitude of decrease was dose dependent (FIGS. 12, 13). The largest effect was observed at the highest concentration of eugenol (60 μL/L), which induced maximum frequency drops of 184-277 Hz (median: 232 Hz), compared to the corresponding median baseline frequency determined during the 30 min immediately preceding the anesthesia.

Comparison of the medians of the EOD frequency during the 30 min before the start of the anesthesia, and during the 15 min after the onset of anesthesia (i.e., including the 5 min of the fish's exposure to the eugenol solution and the first 10 min after their return to the respective home tank) revealed a significant difference, in response to eugenol, whereas the corresponding control experiments had no significant effect (Related Samples Sign Test, n=8 fish; for details see Table 3).

TABLE 3
Difference in median EOD frequency change between the 30-min interval before and
the 15-min interval after the start of anesthesia. Significant differences are indicated by *.

| | Control | 30 μL/L | 45 μL/L | 60 μL/L |
|---|---|---|---|---|
| p-value | 0.2891 | 0.0078* | 0.0078* | 0.0078* |
| Median (Hz) | 1.16 | −48.18 | −65.54 | −88.65 |
| Range (Hz) | [−4.68, 12.15] | [−65.83, −33.57] | [−92.80, −58.62] | [−121.68, −52.50] |

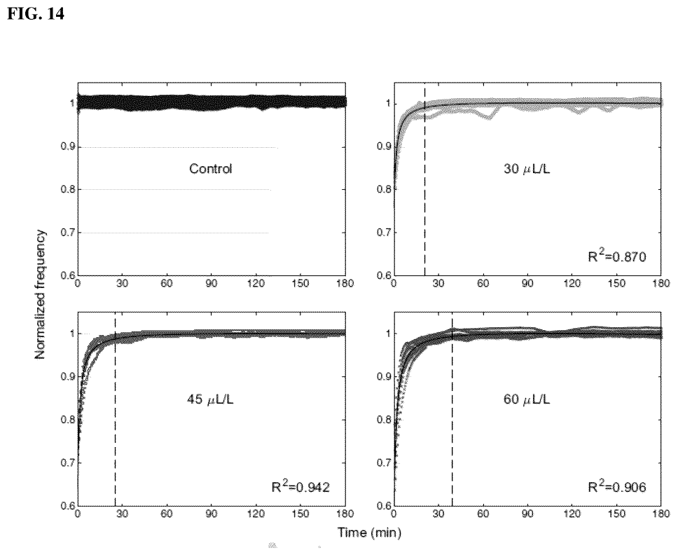

After transfer of the anesthetized fish back to their home tanks, the EOD frequency returned to baseline levels within approximately 30 min (FIG. 14). The time course of recovery followed the model described by Eq. 3 (see black curves in FIG. 14; see Table 4 for fitted model parameters). Comparisons of the maximum decrease in normalized frequency (with respect to baseline EOD frequency) revealed significant differences between different eugenol concentrations (p=0.008, Related Samples Sign Test, n=8 fish; for details see FIG. 14 and Table 5).

TABLE 4
Parameters identified for the model described by Eq. 3, using
nonlinear regression onn ormalized frequency data points
associated with the recovery part of eugenol anesthesia
(cf. legend of FIG. 14).

| Parameter | | | |
|---|---|---|---|
| (unit) | 30 μL/L | 45 μL/L | 60 μL/L |
| a (1) | 0.7465 | 0.6344 | 0.5649 |
| b (min) | 1.4568 | 1.4064 | 1.5816 |
| c (1) | 1.0089 | 1.0084 | 1.0131 |
| d (min⁻¹) | −3.2299 × 10⁻⁵ | −3.3821 × 10⁻⁵ | −6.2321 × 10⁻⁵ |

TABLE 5
Difference in the largest normalized EOD frequency drops induced
by different treatment protocols.

| | 30 μL/L vs. Control | 45 μL/L vs. 30 μL/L | 60 μL/L vs. 45 μL/L |
|---|---|---|---|
| p-value | 0.0078* | 0.0078* | 0.0078* |
| Median | −0.1650 | −0.0715 | −0.0349 |
| Range | [−0.2202, −0.1262] | [−0.1231, −0.0169] | [−0.1000, −0.0056] |

### Effect of Eugenol Anesthesia on Chirp Production Rate

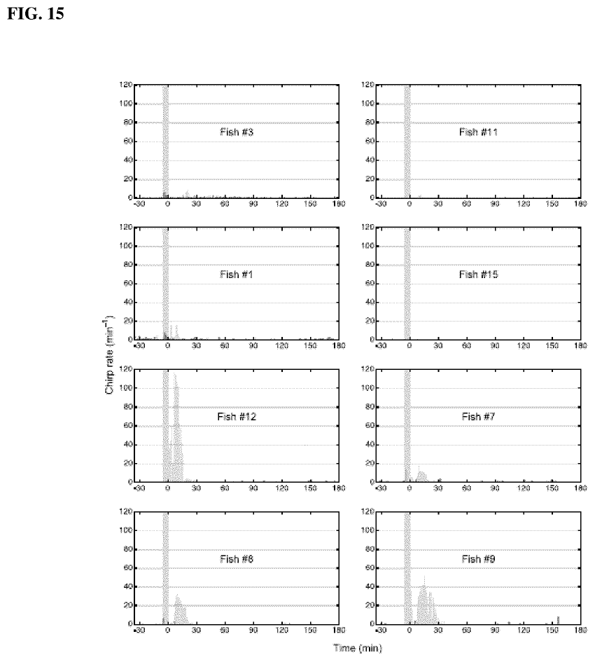

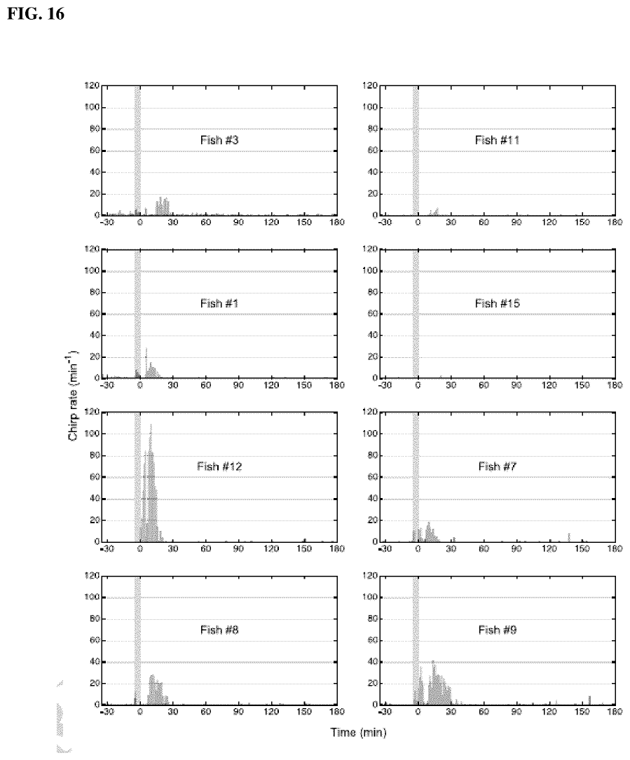

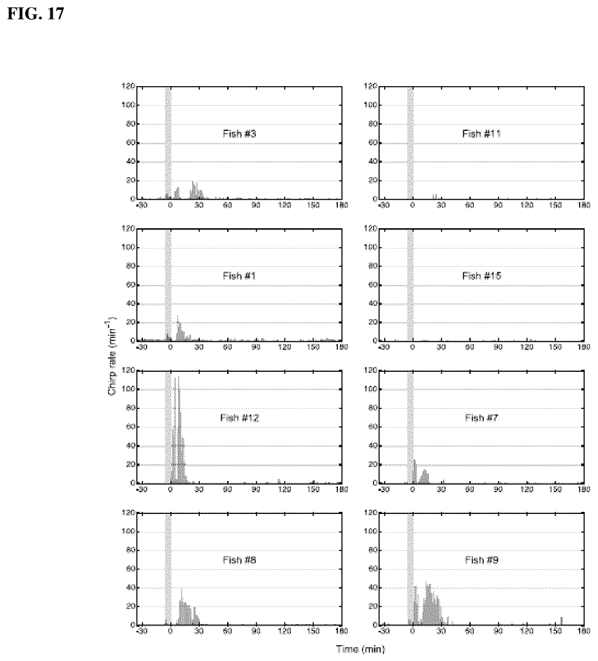

Chirp rates were very low prior to anesthesia at any of three eugenol concentrations or to the control treatment (range: 0-1.57 chirps/min; median: 0.05 chirps/min; n=32 pre-treatment EOD recordings over 30 min each, based on 4 pre-treatment recordings in each of the 8 fish) (FIGS. 15-17).

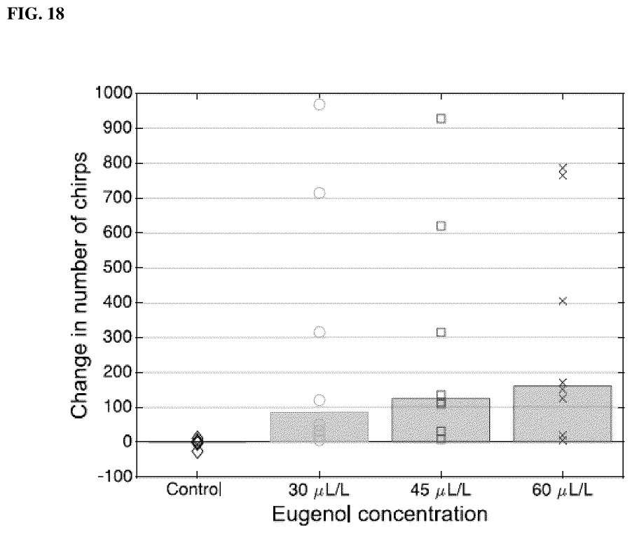

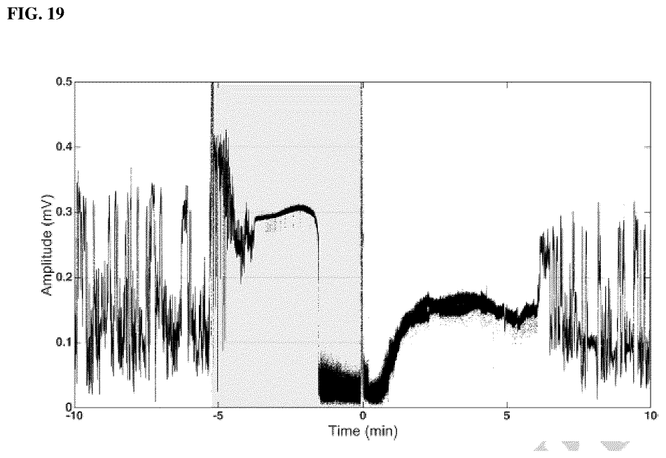

In each of the 8 fish, eugenol induced an increase in the number of chirps produced. This effect was largely restricted to the 30 min immediately following the 5-min exposure of the fish to the anesthetic. During this period, at each of the three eugenol concentrations tested, the increase in the number of chirps compared to the 30 min before anesthesia was significantly higher than for the control treatment (p=0.008, Related-Samples Sign Test, n=8 fish) (FIGS. 15-18, Table 6). However, the magnitude of the increase in chirp rate was independent of the concentration of eugenol used (FIGS. 15-18; Table 7).

TABLE 6
Difference in increase in the number of chirps induced by
each of the three eugenol concentrations versus control.
Changes in chirp rate were compared between the
30-min interval before anesthesia and the 30-min
interval after exposure to the anesthetic.

| | 30 μL/L vs. | 45 μL/L vs. | 60 μL/L vs. |
|---|---|---|---|
| | Control | Control | Control |
| p-value | 0.0078* | 0.0078* | 0.0078* |
| Median | 97.5 | 137 | 160 |
| Range | [9, 969] | [11, 928] | [8, 786] |

TABLE 7
Difference in increase in the number of chirps induced by
anesthesia between pairs of different eugenol concentrations.
Changes in chirp rate were compared between the 30-min
interval before anesthesia and the 30-min interval after
exposure to the anesthetic.

| | 45 μL/L vs. | 60 μL/L vs. | 60 μL/L vs. |
|---|---|---|---|
| | 30 μL/L | 45 μL/L | 30 μL/L |
| p-value | 0.7266 | 0.7266 | 0.2891 |
| Median | 8 | 23 | 60.5 |
| Range | [−95, 77] | [−163, 166] | [−204, 119] |

### Effect of Eugenol Anesthesia on EOD Amplitude

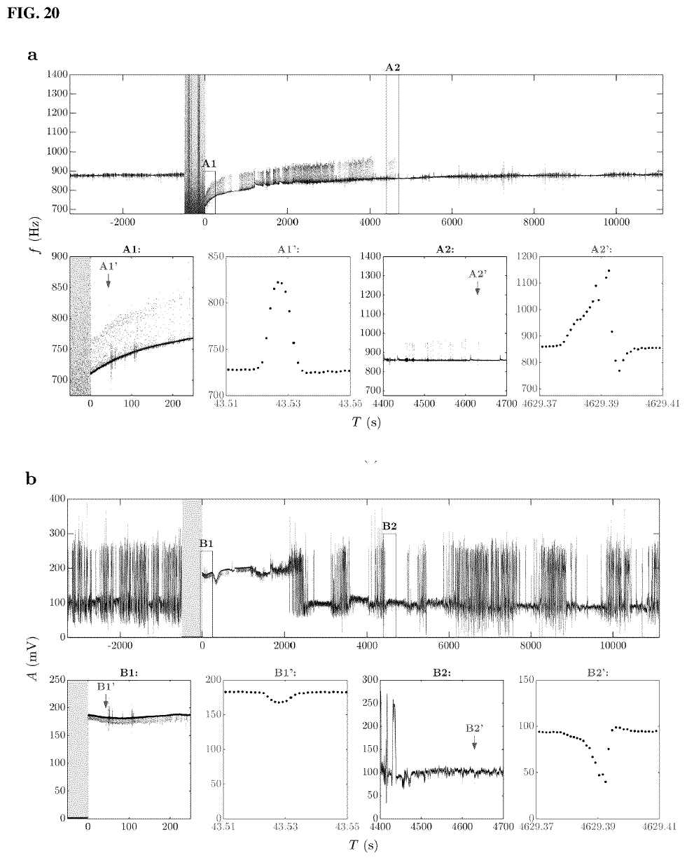

In 4 out of 8 fish exposed to 60 μL/L eugenol, the EOD amplitude collapsed to markedly lower levels approximately 3.5 min after the onset of anesthesia (FIG. 19). During the remaining time of the fish's exposure to the anesthetic, the amplitude either remained at this low level (in 1 fish) or even decreased (in 3 fish). After transferring the fish back to its home tank, the EOD amplitude gradually returned to baseline levels within a few minutes.

### Discussion

### Eugenol as a Fish Anesthetic

Clove oil and its active ingredients have been tested as sedatives and anesthetics in numerous fish species. Although their use in fish intended for human consumption is not approved in some countries (such as the United States; FDA 2007), or is conditional upon compliance with maximum residue limits in foodstuffs in other countries (such as the member states of the European Union; European Commission 2011), these compounds have become increasingly common over the last few decades for anesthesia of ornamental fish and fish used in research.

In the present study, it was found that, in A. leptorhynchus, concentrations of 45 μL/L and 60 μl/L eugenol induce anesthesia characterized by total loss of equilibrium and cessation of any movement, including undulation of the anal fin, within 1-4 min. This result is comparable to observations made in two other small-sized tropical/subtropical freshwater fish, zebrafish (Danio rerio) and red garra (Garra rufa). Both species belong to the same teleostean superorder (Ostariophysi) as A. leptorhynchus. In D. rerio, a stage of anesthesia similar to the one induced in A. leptorhynchus was achieved with a concentration of 60 μL/L eugenol within 1.5 min. In G. rufa, total loss of equilibrium and movement, as well as absence of reaction to external stimuli, was observed at eugenol concentrations of 25 μL/L and 50 μL/L after approximately 3 and 1.5 min, respectively.

In the latter investigation, it was furthermore shown that induction time significantly decreases with stepwise increases in eugenol concentrations from 12.5 μL/L to 150 μL/L. This effect is reminiscent of our finding in A. leptorhynchus of a dose-dependent decrease in time at which locomotor activity ceases.

### Effects of Eugenol on CNS Function

### High Efficacy in Crossing the Blood-Brain Barrier

At the eugenol concentrations used in the present study (30-60 μL/L), the fish started to lower the EOD frequency 1-2 min after their transfer into the anesthetic solution. This very short latency is comparable to the latencies observed in a previous investigation when the fish were anesthetized with MS-222 or urethane. However, the latter two anesthetics required significantly higher concentrations for inducing frequency decreases after similar latencies—200 mg/L in the case of MS-222 and 25 g/L in the case of urethane. Since a critical step toward exerting the frequency-lowering effects in the Pn is the ability of anesthetics to cross the blood-brain barrier, it appears reasonable to assume that the efficacy of eugenol in this step is higher than those of MS-222 and urethane. As is well established in clinical practice, almost all drugs that interfere with brain function are lipid soluble and small, with a molecular mass<400 Da. Each of the three anesthetics used in our two studies meet the latter criterion (molecular masses: eugenol, 164 g/mol; MS-222, 261 g/mol; urethane, 89 g/mol). Unfortunately, the octanol-water partition coefficients are available only for eugenol (log k_{ow}=2.49) (National Center for Biotechnology Information 2023a) and urethane (log k_{ow}=−0.15). However, these data support our hypothesis that, while the solubility of eugenol in water is sufficient for dissolving the compound at the concentrations used in the present study), its lipophilicity enables the molecule to cross the blood-brain barrier through lipid-mediated transport rapidly and with high efficacy.

### Comparative Aspects of the Effect of Eugenol on CNS Function

Despite the wide use of eugenol and related compounds as anesthetics in fish, little is known about their effects on CNS function. A first attempt to fill this critical gap in knowledge was undertaken recently by employing an antidromic-stimulation regime of the axon of the Mauthner cell to study the effect of different concentrations of isoeugenol on the generation and propagation of action potentials in goldfish (Carassius auratus). No effect was observed at 10 mg/L and 20 mg/L, even after exposure of the fish to the anesthetic for more than 1 hour. At 40 mg/L isocugenol, a slight decrease in the slope of action potentials was evident after 10-30 min. Only at the highest concentration tested, 60 mg/L, and exposure of the fish for at least 10-30 min, additional aspects of the action potential were affected, including peak amplitude and duration. However, even the latter changes were rather modest, compared to controls. By contrast, sensory stimulation by brief acoustic or visual pulses had more pronounced effects on postsynaptic potentials induced in the Mauthner neuron, with visual stimuli evoking stronger responses than acoustic stimuli. The authors interpreted these results as evidence that isocugenol acts primarily locally on sensory systems, instead of affecting action potential generation and propagation, as well as chemical and electric transmission, at the level of central neurons. Nonetheless, the limitation of their study to just one neuron, and the lack of experiments examining possible effects of isoeugenol on synaptic transmission, hamper such generalization.

In contrast to this study, the findings of the present investigation demonstrate that eugenol can induce strong dose-dependent effects on the function of a brain system, even at low concentration and within a few minutes. These observations are consistent with the results of experiments carried out on rat neocortical and hippocampal slices that demonstrated reversible and dose-dependent suppression by eugenol of epileptiform field potentials and spreading depression, presumably via inhibition of synaptic plasticity. On the other hand, it is important to keep in mind that direct comparison of our study and that of Machnik et al. (2023) is not possible because not only were different fish species (A. leptorhynchus vs. C. auratus) and different neural systems (pacemaker nucleus vs. Mauthner neuron) examined but also different, though closely related, agents (eugenol vs. isocugenol) were employed.

### Fast Recovery of EOF Frequency After Eugenol Anesthesia

Assaying the EOD frequency as a proxy of the output frequency of the Pn enabled us not only to analyze, with high temporal resolution, the drop in frequency induced by cugenol, but also to track the recovery of the EOD frequency after the fish's return from the anesthetic solution to the water of its home tank. Although the fish regained equilibrium and locomotor activity, including undulation of the anal fin, within a few minutes (data not shown), the EOD frequency remained reduced significantly longer, for up to 30 min. Both the drop in EOD frequency during anesthesia and the persistence of this effect beyond the immediate state of anesthesia underscore the need to consider the possibility of effects on CNS function whenever neurophysiological experiments on fish are performed under anesthesia with eugenol (or related compounds).

Compared to fish anesthetics tested previously by employing the neuro-behavioral assay, it is notable that the recovery time after eugenol anesthesia is markedly shorter than the recovery time of urethane (approximately 3 hours) and MS-222 (approximately 1 hour). Thus, the relatively short ‘neural’ recovery period of eugenol is another attractive feature of this anesthetic, in addition to the advantages mentioned in section ‘Eugenol as a fish anesthetic,’ above.

### Molecular and Cellular Mechanisms of the Anesthetic Effect of Eugenol on the EOD in A. leptorhynchus: a Working Hypothesis

### A Mechanistic Model for Explaining the Effect of Eugenol on EOD Frequency but not on Chirping Behavior

In the following, what is known about the pharmacological actions of eugenol to the neural circuitry involved in generating and modulating the output frequency of the pacemaker nucleus, thereby providing a mechanistic model of the effect of this anesthetic on the EOD frequency was compared. A similar model for explaining the effect of eugenol on chirping behavior was not presented. The generation of chirps by the pacemaker nucleus is primarily controlled by a projection to its relay cells, originating in a subnucleus of the central posterior/prepacemaker nucleus, the so-called CP/PPn-C. This control is exerted by glutamatergic synaptic transmission and activation of non-NMDA-type receptors. The CP/PPn-C, in turn, receives a multitude of inputs from other brain regions. However, unlike the connection between the CP/PPn-C and the relay cells, the nature (excitatory, inhibitory) of these inputs to the CP/PPn-C, and the transmitters/receptors involved at their synapses, are unknown. We, therefore, can only speculate that one or more of these inputs are targets of eugenol, and it is this action that results in the increase in chirping.

### Eugenol Limits the Generation of Action Potentials

Several cellular and molecular effects of eugenol have been implicated in the anesthetic activity of eugenol. Notably, its ability to block the generation of nerve impulses and to reduce the excitability of neurons has been postulated to underlie its property as a local and general anesthetic. Possible molecular targets of this action are voltage-gated sodium channels, which are inhibited by eugenol. In Naᵥ 1.7-transfected CHO cells, this molecule preferentially binds to such channels in the inactivated state, thereby driving fast-inactivated channels into slow-inactivated channels. In addition, methyl-eugenol has been shown to exert inhibitory effects on Kv1.5 and, possibly, other potassium channels. The resulting inhibition of K⁺ currents may lead to clongation of the duration of action potentials and the refractory period.

In the Pn of A. leptorhynchus, the inhibitory effects of cugenol on voltage-gated sodium channels and voltage-gated potassium channels may limit the generation of action potentials and result in the prolongation of action-potentials, respectively. It was proposed that the combination of these effects reduces the frequency of the sustained high-frequency oscillations of the Pn. This hypothesis is congruent with experimental evidence and predictions obtained through modeling, which have suggested the extracellular potassium concentration and the potassium channel conductances as critical determinants of the frequency of the spontaneous oscillations of this brainstem nucleus. The notion that the effect of anesthetics, including MS-222 and urethane as well as eugenol, is mediated by the action of these anesthetics directly in the Pn, but not by modulation of afferent input, receives strong support by electrophysiological recording from isolated Pn tissue.

### Eugenol Potentiates the GABA_{A}-Receptor Response

A second mechanisms that may contribute to the effect of eugenol to induce a drop in EOD frequency is its ability to potentiate GABAA-receptor response. Such an effect has been observed in test systems based on GABAA receptors expressed in Xenopus oocytes and is consistent with the results obtained in a [³H] muscimol-binding assay using rat cortical homogenate. A different effect of eugenol was observed in a study that examined how cugenol affects GABA-induced current in rat trigeminal ganglia and in human embryonic kidney 293 cells expressing the GABA_{A} receptor subtype α1β2γ2. The authors found that it inhibits GABA_{A} current in a reversible, dose-dependent manner. The reason for the discrepancy between the studies of Aoshima and Hamamoto (1999), Sahin et al. (2017), and Kheawfu et al. 2022) on the one hand, and of Lee et al. (2015) on the other, is unclear. Since GABA application to the SPPn lowers the frequency of the EOD, and thus the output frequency of the Pn, a potentiation of the response of GABA receptors received by input from the nE↓ would be expected to result in a decrease of the oscillation frequency of the Pn.

A similar hypothesis related to the potentiation of the inhibitory function of GABA_{A} receptors has been suggested as a mechanistic explanation for the drop in EOD frequency caused by urethane but not MS-222. Remarkably, the time courses of the frequency change during the fish's exposure to eugenol, as shown in the present study, and urethane resemble each other strikingly but both differ significantly from such a plot obtained after MS-222 anesthesia. It is hypothesized that such differences in the ‘signatures’ of the time-frequency plots established through the neuro-behavioral assay reflect differences in the cellular and molecular mechanisms mediating the reduction in EOD frequency.

### REFERENCES CITED

Aoshima H, Hamamoto K (1999) Potentiation of GABA_{A} receptors expressed in Xenopus oocytes by perfume and phytoncid. Biosci Biotechnol Biochem 63:743-748. https://doi.org/10.1271/bbb.63.743
Aydin B (2022) Anaesthetic efficacy of eugenol in doctor fish (Garra rufa): behavioural and cardiovascular responses. Aquac Res 53:6419-6429. https://doi.org/10.1111/are.15049
Bennett M V L (1971) Electric organs. In: Hoar W S, Randall D J (eds) Fish Physiology, Vol 5: Sensory Systems and Electric Organs Academic Press, New York, pp 347-491
Brodin P, Røed A (1984) Effects of eugenol on rat phrenic nerve and phrenic nerve-diaphragm preparations. Arch Oral Biol 29:611-615. https://doi.org/10.1016/0003-9969(84)90130-4
Chung G, Oh S B (2013) Eugenol as local anesthetic. In: Ramawat K G, Mérillon J-M (eds) Natural products: phytochemistry, botany and metabolism of alkaloids, phenolics and terpenes. Springer-Verlag, Berlin, Heidelberg, pp 4001-4015
de Oliveira-Castro G (1955) Differentiated nervous fibers that constitute the electric organ of Sternarchus albifrons, Linn. An Acad Bras Cienc 27:557-564.
Dye J (1991) Ionic and synaptic mechanisms underlying a brainstem oscillator: an in vitro study of the pacemaker nucleus of Apteronotus. J Comp Physiol A 168:521-532. https://doi.org/10.1007/BF00215074
Dye J, Heiligenberg W (1987) Intracellular recording in the medullary pacemaker nucleus of the weakly electric fish, Apteronotus, during modulatory behaviors. J Comp Physiol A 161:187-200. https://doi.org/10.1007/BF00615240
Dye J C, Meyer J H (1986) Central control of the electric organ discharge in weakly electric fish. In: Bullock T H, Heiligenberg W (eds) Electroreception. John Wiley & Sons, New York, pp 71-102
Elekes K, Szabo T (1985) Synaptology of the medullary command (pacemaker) nucleus of the weakly electric fish (Apteronotus leptorhynchus) with particular reference to comparative aspects. Exp Brain Res 60:509-520. https://doi.org/10.1007/BF00236936
Engler G, Fogarty C M, Banks J R, Zupanc G K H (2000) Spontaneous modulations of the electric organ discharge in the weakly electric fish, Apteronotus leptorhynchus: a biophysical and behavioral analysis. J Comp Physiol A 186:645-660. https://doi.org/10.1007/s003590000118
Engler G, Zupanc G K H (2001) Differential production of chirping behavior evoked by electrical stimulation of the weakly electric fish, Apteronotus leptorhynchus. J Comp Physiol A 187:747-756. https://doi.org/10.1007/s00359-001-0248-8
Eske A I, Lehotzky D, Ahmed M, Zupanc G K H (2023) The effect of urethane and MS-222 anesthesia on the electric organ discharge of the weakly electric fish Apteronotus leptorhynchus. J Comp Physiol A 209:437-457. https://doi.org/10.1007/s00359-022-01606-6
European Commission (2011) Commission Regulation (EU) No 363/2011 of 13 Apr. 2011 amending the Annex to Regulation (EU) No 37/2010 on pharmacologically active substances and their classification regarding maximum residue limits in foodstuffs of animal origin, as regards the substance isocugenol. European Commission, Brussels FDA (2007) Concerns related to the use of clove oil as an anesthetic for fish. CVM GFI #150. U.S. Department of Health and Human Services, Food and Drug Administration, Center for Veterinary Medicine, Rockville, MD
Grush J, Noakes D L G, Moccia R D (2004) The efficacy of clove oil as an anesthetic for the zebrafish, Danio rerio (Hamilton). Zebrafish 1:46-53. https://doi.org/10.1089/154585404774101671
Hartman D, Lehotzky D, Ilieş I, Levi M, Zupanc G K H (2021) Modeling of sustained spontaneous network oscillations of a sexually dimorphic brainstem nucleus: the role of potassium equilibrium potential. J Comput Neurosci 49:419-439. https://doi.org/10.1007/s10827-021-00789-2
Heiligenberg W, Metzner W, Wong C J H, Keller C H (1996) Motor control of the jamming avoidance response of Apteronotus leptorhynchus: evolutionary changes of a behavior and its neuronal substrates. J Comp Physiol A 179:653-674. https://doi.org/10.1007/BF00216130
Ilieş I, Zupanc G K H (2023) Computational modeling predicts regulation of central pattern generator oscillations by size and density of the underlying heterogenous network. J Comput Neurosci 51 87-105. https://doi.org/10.1007/s10827-022-00835-7
Kassambara A (2023) Pipe-friendly framework for basic statistical test. R package version 0.7.2. Available from https://rpkgs.datanovia.com/rstatix/. Accessed on 25 Apr. 2023
Kheawfu K, Pikulkaew S, Wellendorph P, Jørgensen LvG, Rades T, Müllertz A, Okonogi S (2022) Elucidating pathway and anesthetic mechanism of action of clove oil nanoformulations in fish. Pharmaceutics 14:919. https://doi.org/10.3390/pharmaceutics 14050919
Kozam G (1977) The effect of eugenol on nerve transmission. Oral Surg Oral Med Oral Pathol 44:799-805. https://doi.org/10.1016/0030-4220(77)90390-5
Kuznetsova A, Brockhoff P B, Christensen R H B (2017) ImerTest Package: tests in linear mixed effects models. J Stat Softw 82:1 - 26. https://doi.org/10.18637/jss.v082.i13
Lee S H, Moon J Y, Jung S J, Kang J G, Choi S P, Jang J H (2015) Eugenol inhibits the GABA_{A} current in trigeminal ganglion neurons. PLOS One 10:00117316. https://doi.org/10.1371/journal.pone.0117316
Li H Y, Park C-K, Jung S J, Choi S-Y, Lee S J, Park K, Kim J S, Oh S B (2007) Eugenol inhibits K⁺ currents in trigeminal ganglion neurons. J Dent Res 86:898-902. https://doi.org/10.1177/154405910708600918
Machnik P, Biazar N, Schuster S (2023) Recordings in an integrating central neuron reveal the mode of action of isocugenol. Commun Biol 6:309. https://doi.org/10.1038/s42003-023-04695-4
Metzner W (1999) Neural circuitry for communication and jamming avoidance in gymnotiform electric fish. J Exp Biol 202:1365-1375. https://doi.org/10.1242/jeb.202.10.1365
Meyer J H (1984) Steroid influences upon discharge frequencies of intact and isolated pacemakers of weakly electric fish. J Comp Physiol A 154:659-668. https://doi.org/10.1007/BF01350219
Meyer J H, Leong M, Keller C H (1987) Hormone-induced and maturational changes in electric organ discharges and electroreceptor tuning in the weakly electric fish Apteronotus. J Comp Physiol A 160:385-394. https://doi.org/10.1007/BF00613028
Moortgat K T, Bullock T H, Sejnowski T J (2000) Gap junction effects on precision and frequency of a model pacemaker network. J Neurophysiol 83:984-997. https://doi.org/10.1152/jn.2000.83.2.984
Moreira-Lobo D C A, Linhares-Siqueira E D, Cruz G M P, Cruz J S, Carvalho-de-Souza J L, Lahlou S, Coelho-de-Souza A N, Barbosa R, Magalhães P J C, Leal-Cardoso J H (2010) Eugenol modifies the excitability of rat sciatic nerve and superior cervical ganglion neurons. Neurosci Lett 472:220-224. https://doi.org/10.1016/j.neulet.2010.02.009
Müller M, Pape H-C, Speckmann E-J, Gorji A (2006) Effect of eugenol on spreading depression and epileptiform discharges in rat neocortical and hippocampal tissues. Neuroscience 140:743-751. https://doi.org/10.1016/j.neuroscience.2006.02.036
National Center for Biotechnology Information (2023a) PubChem Compound Summary for CID 3314, Eugenol. Available from https://pubchem.ncbi.nlm.nih.gov/compound/Eugenol. Accessed on Sep. 27, 2023
National Center for Biotechnology Information (2023b) PubChem Compound Summary for CID 5641, Urethane. Available from https://pubchem.ncbi.nlm.nih.gov/compound/Urethane. Accessed on Sep. 27, 2023
Neiffer D L, Stamper M A (2009) Fish sedation, analgesia, anesthesia, and euthanasia: considerations, methods, and types of drugs. ILAR J 50:343-360. https://doi.org/10.1093/ilar.50.4.343
Pardridge W M (2012) Drug Transport across the Blood-Brain Barrier. J Cereb Blood Flow Metab 32:1959-1972. https://doi.org/10.1038/jcbfm.2012.126
Park C-K, Kim K, Jung S J, Kim M J, Ahn D K, Hong S-D, Kim J S, Oh S B (2009) Molecular mechanism for local anesthetic action of eugenol in the rat trigeminal system. Pain 144:84-94. https://doi.org/10.1016/j.pain.2009.03.016
Priborsky J, Velisek J (2018) A review of three commonly used fish anesthetics. Rev Fish Sci Aquac 26:417-442. https://doi.org/10.1080/23308249.2018.1442812
Sahin S, Eulenburg V, Heinlein A, Villmann C, Pischetsrieder M (2017) Identification of eugenol as the major determinant of GABA_{A}-receptor activation by aqueous Syzygium aromaticum L. (clove buds) extract. J Funct Foods 37:641-649. https://doi.org/10.1016/j.jff.2017.08.033
Schaefer J E, Zakon H H (1996) Opposing actions of androgen and estrogen on in vitro firing frequency of neuronal oscillators in the electromotor system. J Neurosci 16:2860-2868. https://doi.org/10.1523/jneurosci.16-08-02860.1996
Sîrbulescu R F, Ilieş I, Zupanc G K H (2014) Quantitative analysis reveals dominance of gliogenesis over neurogenesis in an adult brainstem oscillator. Dev Neurobiol 74:934-952. https://doi.org/10.1002/dneu.22176
Trowbridge H, Edwall L, Panopoulos P (1982) Effect of zinc oxide-eugenol and calcium hydroxide on intradental nerve activity. J Endod 8:403-406. https://doi.org/10.1016/S0099-2399(82)80094-0
Wang Z-J, Tabakoff B, Levinson S R, Heinbockel T (2015) Inhibition of Naᵥ1.7 channels by methyl eugenol as a mechanism underlying its antinociceptive and anesthetic actions. Acta Pharmacol Sin 36:791-799. https://doi.org/10.1038/aps.2015.26
Waxman S G, Pappas G D, Bennett M V L (1972) Morphological correlates of functional differentiation of nodes of Ranvier along single fibers in the neurogenic electric organ of the knife fish Sternarchus. J Cell Biol 53:210-224. https://doi.org/10.1083/jcb.53.1.210
Yalkowsky S H, He Y, Jain P (2010) Handbook of aqueous solubility data. 2nd edition. CRC Press, Boca Raton
Zupanc G K H (2002) From oscillators to modulators: behavioral and neural control of modulations of the electric organ discharge in the gymnotiform fish, Apteronotus leptorhynchus. J Physiol Paris 96:459-472. https://doi.org/10.1016/S0928-4257(03)00002-0
Zupanc G K H (2020) Development of a sexual dimorphism in a central pattern generator driving a rhythmic behavior: The role of glia-mediated potassium buffering in the pacemaker nucleus of the weakly electric fish Apteronotus leptorhynchus. Dev Neurobiol 80:6-15. https://doi.org/10.1002/dncu.22736
Zupanc G K H, Amaro S M, Lehotzky D, Zupanc F B, Leung N Y (2019) Glia-mediated modulation of extracellular potassium concentration determines the sexually dimorphic output frequency of a model brainstem oscillator. J Theor Biol 471:117-124. https://doi.org/10.1016/j.jtbi.2019.03.013
Zupanc G K H, Banks J R, Engler G, Beason R C (2003) Temperature dependence of the electric organ discharge in weakly electric fish. In: Ploger B J, Yasukawa K (eds) Exploring animal behavior in laboratory and field: an hypothesis-testing approach to the development, causation, function, and evolution of animal behavior. Academic Press, Amsterdam, pp 85-94
Zupanc G K H, Ilieş I, Sîrbulescu R F, Zupanc M M (2014) Large-scale identification of proteins involved in the development of a sexually dimorphic behavior. J Neurophysiol 111:1646-1654. https://doi.org/10.1152/jn.00750.2013
Zupanc G K H, Maler L (1993) Evoked chirping in the weakly electric fish Apteronotus leptorhynchus: a quantitative biophysical analysis. Can J Zool 71:2301-2310. https://doi.org/10.1139/293-323
Zupanc G K H, Sîrbulescu R F, Nichols A, Ilies I (2006) Electric interactions through chirping behavior in the weakly electric fish, Apteronotus leptorhynchus. J Comp Physiol A 192:159-173. https://doi.org/10.1007/s00359-005-0058-5

### Example 3—Supervised Learning Algorithm for Analysis of Communication Signals in the Weakly Electric Fish Apteronotus leptorhynchus

Signals as vehicles for transmission of information from a sender to a receiver play a pivotal role in animal communication. Broadcasting of signals is mediated by a variety of sensory channels, such as visual, acoustic, tactile, chemical, and electric. Diversity of signals, cither within one sensory modality or by activation of several sensory channels, enables animals to use different signals for different behavioral functions. Within one sensory modality, signal diversity is often achieved by modulation of a generic type of signal. For example, different acoustic signals can be produced by temporal frequency and amplitude modulations, and even rather subtle differences can have profoundly different functional effects.

While acoustic signals are displayed intermittently only (although sometimes for prolonged periods at high rates), some electric fishes produce a generic form of electric signal continuously throughout life. This group includes the brown ghost knifefish (Apteronotus leptorhynchus), a species of the taxonomic order Gymnotiformes that has been intensively studied as model organisms in ethology and neurocthology.

A. leptorhynchus generates such continuous electric discharges with its electric organ composed of modified axonal terminals of spinal motoneurons. The synchronous depolarization of these so-called electrocytes produces electric pulses separated by short inter-pulse intervals. This results in the appearance of a continuous, wave-like signal, commonly referred to as electric organ discharge (EOD). The frequency at which the fish generates the EOD train is determined, in a one-to-one fashion, by the frequency of the neural oscillations of a central pattern generator in the medulla oblongata, the pacemaker nucleus. Within the species-specific frequency range of 650 to 1000 Hz, males discharge at higher frequencies than females, with little overlap between the sexes. Owing to this sexual dimorphism, the EOD contains information about the sex of its sender.

Whereas the species as whole occupies a broad EOD frequency range, the frequency of the discharges of a given individual within this frequency band is highly constant, as indicated by the coefficient of variation (cv=(standard deviation/mean)×100 (%)), which assumes values of less than 0.2% over 30-min. Nevertheless, transient modulations may occur, resulting in diversification of the generic EOD signal. The best-characterized type comprises chirps. In isolated individuals of A. leptorhynchus, chirps are very rarely produced, on average less than once per 10 min. However, during stimulation with the EODs of conspecific fish or with electric signals mimicking such EODs, or after administration of certain drugs, chirp production may increase one-thousand-fold to rates as high as 2 s-1.

Chirps last between some tens and a few hundred milliseconds and involve complex frequency and amplitude modulations. Six distinct chirp types have been identified. They are defined by differences in duration as well as the extent of the frequency and amplitude modulations. Spontaneously produced chirps are predominantly of type 1, whereas most chirps evoked by the EODs of a neighboring fish (or mimics of such electric signals) or by proper pharmacological stimulation belong to
the type-2 category. Both type-1 and type 2-chirps are rather short (duration approximately 20 ms) but distinct in terms of the degree of frequency increase (400 Hz versus 100 Hz) and amplitude reduction (approximately 50% versus<10%). Longer chirps of type 3-6 are, most typically, generated by older individuals and directed to fish of the other sex.

While chirps can be elicited from either sex, at similar rates, through application of pharmacological agents, during electric interaction with conspecifics or in response to electric stimuli mimicking a fish's EOD males chirp at much higher rates than females. In addition, chirps are optimally evoked by electric stimuli with frequencies within±10 Hz of the fish's EOD frequency. Thus, type-2 chirps are typically exchanged by males. Moreover, the chirps produced by two electrically interacting fish are not independent of each other. Instead, the chirps generated by one fish follow the chirps of the other individual with a preferred latency of roughly 500-1000 ms. This ‘echo response’ may serve a communicatory function during social interactions, such as aggressive encounters.

Traditionally, different chirp types have been identified and quantified by visual inspection of time-voltage and time-frequency plots. In addition, threshold-based algorithms and a method based on assumed chirp waveform have been used for chirp detection. Whereas these approaches can be successfully employed for the identification of pre-defined chirp types, the definition of chirp categories is subject to the investigator's bias. Moreover, such approaches do not allow detection of possible additional chirp types that remained unnoticed previously.

To address these deficiencies, we have, in the present study, a supervised learning algorithm is developed. Supervised learning is a machine learning paradigm used across many disciplines. Its goal is to learn, from a “ground truth” (GT) data set, a function that assigns proper outputs (in the present study: time instances of chirps and associated chirp types) to inputs (in the present study: time-series frequency and amplitude data). While the suitability of this machine learning paradigm for the unbiased analysis of chirps produced by A. leptorhynchus is demonstrated, it is proposed that similar approaches can be successfully applied to signal analysis in a variety of other ethological and neurocthological systems.

### Materials and Methods

### EOD Recording

For the present investigation, time-voltage recordings of the EOD containing chirps generated spontaneously or evoked pharmacologically were analyzed. These data had been collected as part of a previous study examining the effect of urethane anesthesia on EOD frequency and chirping behavior in A. leptorhynchus. Eight fish (total lengths: median, 116 mm; range, 107-143 mm; body weights: median, 2.9 g; range, 2.5-4.8 g) were used. Their EOD baseline frequencies varied between 683 Hz and 868 Hz (normalized to frequency values expected at 26° C., using a Q10 of 1.56). The morphological data and EOD frequencies indicate that the fish were approximately 1 year old and included both males and females. Details of the experiments and the recording technique are given in Eske et al (2023). Briefly, each fish was kept in an isolation tank in which a cylindrical plastic tube provided shelter. Differential recording of the fish's EOD was done through a pair of stainless-steel electrodes mounted on the inside of the tube. During recording, the two open ends of the tube were closed with a coarse plastic mesh netting to ensure that the fish did not leave the tube. The EOD of each fish was recorded for 30 min before, and 180 min immediately after, general anesthesia. State of anesthesia was induced by transferring the fish into a glass beaker containing 2.5% urethane dissolved in water from the fish's isolation tank. During the pre-anesthesia session, spontaneous chirps occurred but at very low rates of approximately 1 chirp/30 min. Anesthesia induced a tremendous increase in chirping behavior, resulting, on average, in 1500 chirps during the 30 min immediately following anesthesia. For the present analysis, the 30-min-pre-anesthesia recordings, and the 180-min-post-anesthesia recordings, of the 8 fish were combined, yielding a total of 1,680 min of EOD recording. Employing the supervised learning algorithm, a total of 30,734 chirps were detected in these combined recordings.

### Calculation of EOD Frequency and Amplitude

The sampled voltage data (tᵢ, vᵢ), i=1, . . . , Mᵥ, were exported from Spike 2 and processed in MATLAB version R2021b. These data were filtered in 3-s windows with 2-s overlap using a bandpass filter with frequency band [0.5, 1.5]×f₀, where the fundamental frequency f₀ in each 3-s window was determined based on the power spectrum of the signal using fast Fourier transform and the “findpeaks” function of MATLAB.

Using linear interpolation, all time instances were computed where the filtered time-series signal (tᵢ, Vᵢ), i=1, . . . , Mᵥ, crosses the time axis towards positive voltage values:

$${t}^{+} = ( {t}_{j ( k )} - \frac{{V}_{j ( k )}}{{V}_{j ( k ) + 1} - {V}_{j ( k )}} ( {t}_{j ( k ) + 1} - {t}_{j ( k )} ) : k = 1 , … , M ) . \qquad ( 5 )$$

Here the tuple of all upward crossings t⁺ contains M=|j| number of elements, with

j=(i: sgn(Vᵢ₊₁)−sgn(Vᵢ)>0,i=1, . . . ,Mᵥ) (6)

being a tuple containing all indices of the filtered time-series data where the voltage changed sign to a positive value. Finally, for each oscillation interval [t⁺(k), t⁺⁽k+1)], k=1, . . . ,M−1, time instance Tₖ, and associated frequency fₓ and amplitude Aₖ values were computed as

$${T}_{k} = \frac{{t}^{+} ( k + 1 ) - {t}^{+} ( k )}{2} , \\ {f}_{k} = \frac{1}{{t}^{+} ( k + 1 ) - {t}^{+} ( k )} , \\ {A}_{k} = {max}_{j ( k ) \leq i \leq j ( k + 1 )} ( {v}_{i} ) - {min}_{j ( k ) \leq i \leq j ( k + 1 )} ( {v}_{i} ) . \qquad ( 7 - 9 )$$

An example of computed time-series data of frequency and amplitude is shown in FIG. 20.

Chirp detection by supervised learning

### “Ground Truth” Data Set

### Data Collection

Tuples of equal-time-length time-series data segments

Sₙ [symbol] ₍ᵣ₋₁₎₊ⱼ=({Tₖ^{(r)},fₖ^{(r)},Aₖ^{(r)}}:Tₖ^{(r)}∈[Tₛₜₐᵣₜ+(j−1)ΔT,Tₛₜₐᵣₜ+jΔT]k=1, . . . ,M−1),j=1, . . . n [symbol] _{,} (10)

were collected from each recording r=1, . . . , nᵣ, where nᵣ is the total number of EOD recordings, and superscript □(r) indicates association with recording r. The time length of segments was determined as ΔT=(T_{end}−Tₛₜₐᵣₜ)/nₛ. The values of parameters Tstart, Tend, ns, nr, used for the generation of time-series data segments are provided in Table 8.

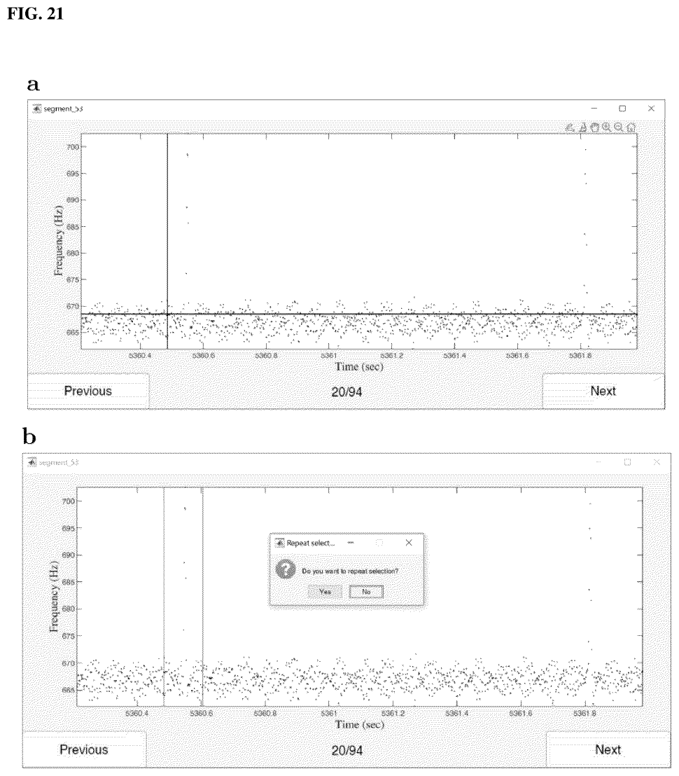

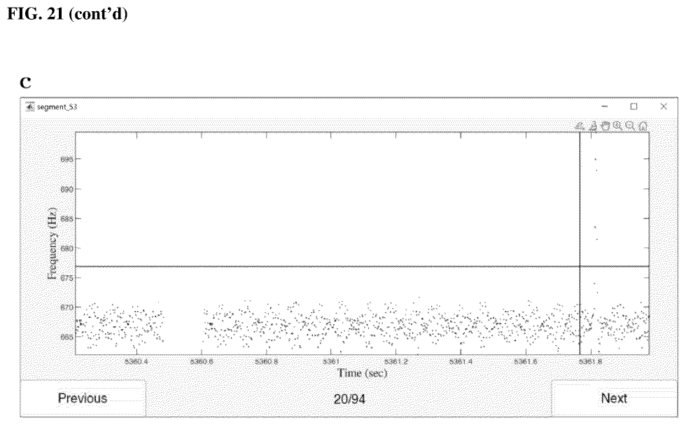

Using the MATLAB tool shown in FIG. 21, a person previously trained to identify chirps collected all chirp instances from each segment Sᵢ for all indices i∈i_{GT}, where the elements of subset iGT⊂{1, . . . , nₛnᵣ}, with n_{GT}=|i_{GT}| (see Table 8), were randomly chosen, without replacement. Although for each data point only time and frequency values were displayed during data collection (see FIG. 21), the associated amplitude values were also stored in the GT set of chirps

G={({T_{i,j},f_{i,j},A_{i,j}}) [symbol] [symbol] } [symbol] ⁿ, (11)

where {T_{i,j}, f_{i,j}, A_{i,j}} is the j-th data point of the i-th GT chirp sample, li denotes the number of data points in the i-th sample, and n is the total number of samples.

### Data Processing

The person who collected chirp samples was instructed to include, in each sample, data points prior to and after chirping, associated with the non-modulated, instantaneous “base” frequency of the fish. Hence, it was assumed that each sample includes both pre and post-chirp data points and estimated

TABLE 8
Parameter values used for generating time-series data segments
Si, i ∈ i_{GT}, from which “ground truth” chirp samples G were
collected. Values of Tₛₜₐᵣₜ and T_{end} were measured in time
coordinates fixed to the start of recordings.

| Parameter | Value |
|---|---|
| Tₛₜₐᵣₜ (s) | 3200 |
| T_{end} (s) | 6200 |
| nₛ (1) | 10 |
| nᵣ (1) | 16 |
| n_{GT} | 64 |

the “base” frequency and amplitude of each sample i as

f_{base,t}=median([f_{i,j}] [symbol] [symbol] , [f [symbol] ₋ⱼ₊₁] [symbol] [symbol] ),

A_{base,t}=median([A_{i,j}]_{z,999} [symbol] , [A [symbol] ₋ⱼ₊₁] [symbol] [symbol] ). (12-13)

where n_{med}<min (ₗᵢ/2) is an arbitrarily chosen positive integer which we set to n_{med}=10. Each sample i=1, . . . , n was normalized with respect to the maximum frequency rise according to

$${φ}_{i , j} = \frac{{f}_{i , j} - {f}_{base , i}}{{max}_{j ∈ \{ 1 , … , {l}_{i} \}} ( {f}_{i , j} ) - {f}_{base , i}} , j = 1 , … , {l}_{i} , \qquad ( 14 )$$

and with respect to the base amplitude as

$${a}_{i , j} = \frac{{A}_{i , j} - {A}_{base , i}}{{A}_{base , i}} , j = 1 , … , {l}_{i} . \qquad ( 15 )$$

Then, the time values of each sample were centered according to

$$\begin{matrix}{\tilde{T}}_{i , j} := {T}_{i , j} - {T}_{i , {j}_{cen , i}} , j = 1 , … , {l}_{i} , \\ {j}_{cen , i} = {argmin}_{k} ( \vert {H}_{i , k} - \frac{1}{2} \vert ) , & ( 16 - 17 ) \\ {H}_{i , k} = \frac{{\sum }_{j = 1}^{k} h ( {φ}_{i , j} )}{{\sum }_{j = 1}^{k} h ( {φ}_{i , j} )} , k = 1 , … , {l}_{i} , & ( 18 )\end{matrix}$$

where rectifier

$$h ( {φ}_{i , j} ) = \frac{ln ( 1 + {e}^{δ ( \vert {φ}_{i , j} \vert - {\overline{φ}}_{i} )} )}{δ - δ {\overline{φ}}_{i}} , \qquad ( 19 )$$

with

φ̅ᵢ=4max(sd({φ_{i,k}}ₖ₌₁ⁿ^{_{med}}),sd({φ_{i,l},−k+1}ₖ₌₁ⁿ^{_{med}})), (20)

was applied for the elimination of noise and to highlight “meaningful” parts of the frequency sample. Here sd(·) denotes the standard deviation, φᵢ is the cutoff value of normalized frequency associated with sample i and δ=50 is an arbitrarily chosen smoothing parameter. Using the empirical cumulative distribution Hᵢ,· of rectified frequency values h(φᵢ,·), each sample was trimmed, such that only the data points j within interval \tilde{ }T_{i,j}∈[−3Δ\tilde{ }Tᵢ, 3Δ\tilde{ }Ti] were kept, with

$$Δ {\hat{T}}_{i} = {\hat{T}}_{i , {j}_{i}^{+}} - {\tilde{T}}_{i , {j}_{i}^{-}} , \\ {j}_{i}^{+} = {argmin}_{k ∈ ( 1 , … , {l}_{k} )} ( \vert {H}_{i , k} - 0.9 \vert ) , {j}_{i}^{-} = {argmin}_{k ∈ \{ 1 , … , {l}_{i} \}} ( \vert {H}_{i , k} - 0.1 \vert ) . \qquad ( 21 - 22 )$$

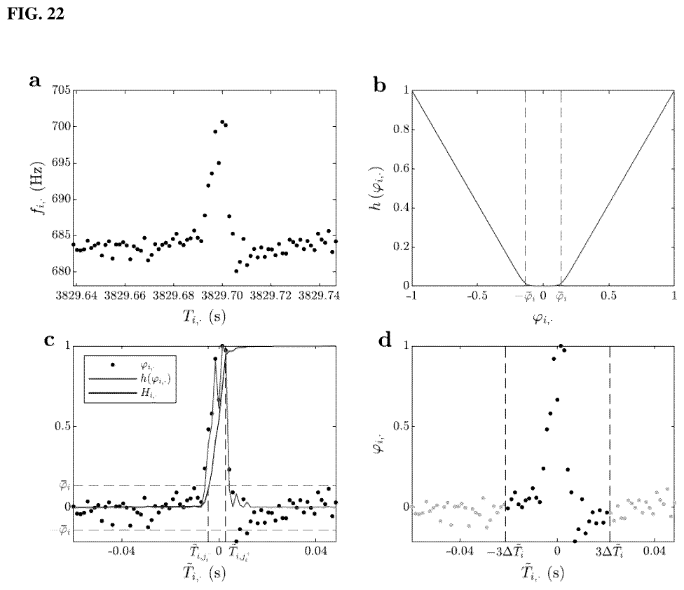

Note that here Δ\tilde{ }Ti is the difference between the 90% and 10% percentile estimates of the empirical cumulative distribution Hi,·. The above described data processing method is illustrated in FIG. 22.

### Grouping and Resampling

Because our supervised learning method requires uniform size among GT samples, we grouped and resampled all GT samples according to the number of data points that formed the individual GT samples.

After trimming, the size of each GT sample was roughly commensurate with the length of the associated chirp. To distinguish between chirps whose duration have different time scales, GT samples were divided into 3 groups and resampled the members of each r group such that associated samples contained 10ᵣ+1 number of points:

Gᵣ={{(T_{i,} [symbol] ₊ⱼ,f_{i,} [symbol] ₊ⱼ,A_{i,} [symbol] ₊ⱼ)}ⱼ₌₋₁₀ [symbol] _{/2}¹⁰ [symbol] ^{/1} : |{\tilde{T}_{i,j}:\tilde{T}_{i,j}∈[−3Δ\tilde{T}ᵢ,3Δ\tilde{T}ᵢ],1≤j≤lᵢ}|∈(10^{r-1}+1,10^{r}+1], 1≤i≤n],r=1,2,3. (23)

Here the fact that all data points inside any GT sample can be located within the associated recording's time-frequency-amplitude data was utilized. For example, if T_{i,1} and T_{q} are from the same recording and that T_{i,1}=T_{q}, then any other point j associated with sample i: (T_{i,j}, f_{i,j},A_{i,j})=(T_{q+j-1}, f_{q+j-1},A_{q+j-1}) can be found.

Note that chirps typically have a duration shorter than 0.5 s, and the highest EOD frequency in A. leptorhynchus is approximately 1000 Hz, therefore GT sample groups Gᵣ, r=1, 2, 3, are able to capture the full length of all chirps.

### Training

### Principal Component Analysis

After resampling, the normalized frequencies and amplitudes (φ_{i,jcen,i}+ⱼ, a_{i,jcen,i}+ⱼ), j=−10^{r}/2, . . . , 10^{r}/2, of each chirp sample i in each GT group Gr were recomputed according to Eqs. 12-15. For ease of notation, in the following, we drop the shift _{jcen,i} in the second subscript index.

For each r, the normalized frequency and amplitude values were collected from Gr

fᵣ⁽ⁱ⁾=[φᵢ−10^{r}/2, . . . , φᵢ,10^{r}/2,]^{T},

aᵣ⁽ⁱ⁾=[αᵢ,−10^{r}/2, . . . ,αᵢ,10^{r}/2]^{T}, (24-25)

of each sample i associated with the training set into a matrix Xᵣ∈R^{mr×2(10r+1)} such that

$${X}_{r}^{T} = [ \begin{matrix}{f}_{r}^{( 1 )} & … & {f}_{r}^{( {m}_{r} )} \\ {a}_{r}^{( 1 )} & … & {a}_{r}^{( {m}_{r} )}\end{matrix} ] , \qquad ( 26 )$$

where mᵣ is the total number of samples in Gᵣ associated with the training set. For the further ease of notation, in the following, index r was dropped, as well. The principal components (PCs) of X were determined by performing spectral decomposition as

X^{T}X=PDP^{T}, (27)

where P=[p₁, . . . , p₂₍₁₀ᵣ₊₁₎] is an orthogonal matrix and D=diag(λ₁, . . . , λ₂₍₁₀ᵣ₊₁₎) is a diagonal matrix, with λ₁≥λ₂≥. . . ≥λ₂₍₁₀ᵣ₊₁₎. Note that here pₖ is the k-th PC with associated empirical variance λₖ. The training data set was then projected onto the space of the first N PCS, i.e.,

Y=XP_{N}, (28)

was computed where P_{N}=[p₁, . . . , p_{N}].

### Gaussian Mixture Model Fitting

The projected data Y^{T}=[y⁽¹⁾, . . . , y^{(m)}] was modeled using the Gaussian mixture model (GMM)

y⁽ⁱ⁾˜ [symbol] (μ_{c},Σ_{c}),c˜M_{C}(p₁, . . . ,p_{C}), (29)

where N(μ_{c},Σ_{c}) is the multivariate normal distribution of the _{c}-th mixture component with mean μ_{c}∈R^{N×1} and covariance Σ_{c}∈R^{N×N}, while M_{C}(p₁, . . . , p_{C}) is a multinomial distribution with C number of categories and mixing proportions p₁, . . . , p_{C}. The unknown parameters were estimated Θ={p₁, . . . , p_{C},μ₁, . . . ,μ_{C},Σ₁, . . . ,Σ_{C}} of this GMM based on data Y using the “fitgmdist” function of MATLAB. Elimination of outliers After fitting the GMM, each data sample i were assigned to the cluster with maximum posterior probability, i.e., the cluster of sample i was computed according to

$${c}_{i} = {argmax}_{c ∈ \{ 1 , … , C \}} ( P ( c \vert i ) ) , \qquad ( 30 )$$

for each i=1, . . . ,m, where P(c|i) is the probability that sample i belongs to cluster c, given the observation y⁽ⁱ⁾. Then, the coefficient of determination (CoD) of the frequency component of each sample was computed with respect to its assigned cluster mean as

$${R}_{i}^{2} = 1 - \frac{{‖ {f}^{( i )} - {\overline{f}}_{{c}_{i}} ‖}^{2}}{{‖ {f}^{( i )} - {\overline{f}}^{( i )} ‖}^{2}} . \qquad ( 31 )$$

Here ∥·∥ denotes the L2 norm and

$${[ {\overline{f}}_{c} , {\overline{a}}_{c} ]}^{T} = {P}_{N} {\hat{μ}}_{c} , \\ {\overline{f}}^{( i )} = \frac{1}{{10}^{r} + 1} ( {1}^{T} {f}^{( i )} ) 1 , \qquad ( 32 - 33 )$$

with 1 being a vector of 1−s.

Each cluster c was eliminated for which the 5% percentile of associated CoD values {Rᵢ²: cᵢ=c, 1≤i≤m} was below threshold δ_{R}²=0.3. Additionally, each cluster c whose size |{i: cᵢ=c,1≤i≤m}| was below threshold δ_{c}=30 was eliminated.

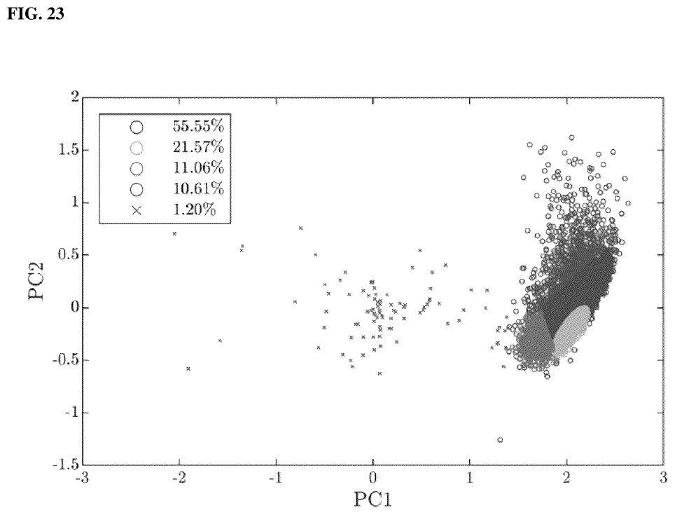

FIG. 23 illustrates the projected training data Y from G₂, with parameters N=2 and C=5; note the eliminated cluster.

### Detection

Training yields PCs P_{N} and GMM

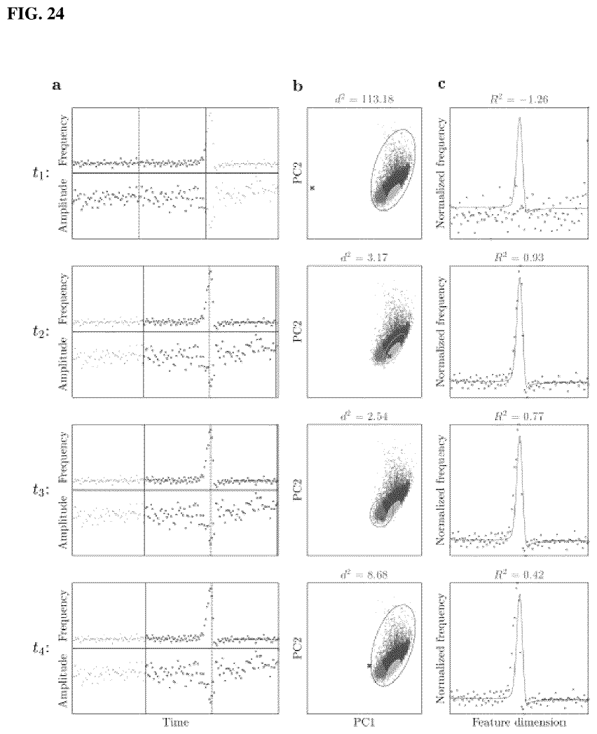

y⁽ⁱ⁾˜ [symbol] (\hat{μ}_{c},\tilde{Σ}_{c}),c˜M_{C} [symbol] (\tilde{p}₁, . . . ,\tilde{p}_{C} [symbol] ), (34)

where C*≤C is the number of kept clusters, with \tilde{ }p_{c}=\hat{ }p_{c}/{Σ_{q=1}^{C*}p_{q}, and \hat{ }p_{c}, \hat{ }μ_{c}, \hat{ }Σ_{c}, being the estimated parameters of kept clusters c=1, . . . , C*. To detect chirps in recordings, data points {(Tᵢ₊ⱼ, fᵢ₊ⱼ,Aᵢ₊ⱼ)}ⱼ₌₀^{10r} i=1, . . . ,M−10^{r}−1, were analyzed in a moving time window containing 10^{r}+samples (see FIG. 24a). At each instance i, normalized frequency and amplitude values were computed

f⁽ⁱ⁾=[φ_{i,1}, . . . ,φ_{i,10}^{r}+1,]^{T},

a⁽ⁱ⁾=[α_{i,1}, . . . , α_{i,10}^{r}+1]^{T}, (35-36)

according to formulas Eqs. 12-15 with (T_{i,j}, f_{i,j},A_{i,j})=(Tᵢ₊ⱼ₋₁, fᵢ₊ⱼ₋₁,Aᵢ₊ⱼ₋₁) and lᵢ=10^{r}+1.

### Mahalanobis-Distance-Based Detection

At each instance i, our Mahalanobis-distance-based (MDB) detection method first projects the normalized frequency and amplitude data onto the PCs according to

$${y}^{( i )} = {P}_{N}^{T} [ \begin{matrix}{f}^{( i )} \\ {a}^{( i )}\end{matrix} ] , \qquad ( 37 )$$

then it determines the kept cluster which is most likely to generate y⁽ⁱ⁾:

$${c}_{i} = {argmax}_{c ∈ \{ 1 , … , {C}^{*} \}} ( P ( c \vert i ) ) . \qquad ( 38 )$$

Afterwards, our method computes the Mahalanobis distance

dᵢ=\sqrt{(y⁽ⁱ⁾−\hat{μ}_{c}_{ᵢ})^{T}\hat{Σ}_{c}_{ᵢ}⁻¹(y⁽ⁱ⁾−\hat{μ}_{c}_{ᵢ})}. (39)

For any point generated by kept cluster cᵢ, realizations dᵢ² follow a chi-squared distribution with N degrees of freedom: Dᵢ²˜ [symbol] _{N}².

The MDB method collects all i instances, where the squared Mahalanobis distance is below threshold ε_{d}² and the maximum frequency rise is above threshold ε_{f}, into the tuple

$${c}_{DMB} = ( i : {d}_{i}^{2} < {ε}_{{d}^{2}} , {max}_{1 \leq j \leq {10}^{r} + 1} ( {f}_{i , j} ) - {f}_{base , i} > {ε}_{f} , i = 1 , … , M - {10}^{r} - 1 ) . \qquad ( 40 )$$

Each contiguous segment in CMDB corresponds to an identified chirp. In each contiguous segment, the identified chirp was associated with the instance i that has lowest distance dᵢ. Threshold ε_{d}² is determined based on a chosen level of significance α such that P(Dᵢ²<ε_{d}²)=1−α. The MDB method is illustrated in FIG. 24b.

### Coefficient-of-Determination-Based Detection

At each instance i, our coefficient-of-determination-based (CDB) detection method computes the CoD of the frequency component with respect to each kept cluster mean according to

$${R}_{i , c}^{2} = 1 - \frac{{‖ {f}^{( i )} - {\overline{f}}_{{c}_{i}} ‖}^{2}}{{‖ {f}^{( i )} - {\overline{f}}^{( i )} ‖}^{2}} , c = 1 , … , {C}^{*} , \qquad ( 41 )$$

using formulae Eqs. 32-33, and assigns instance i to the cluster with highest CoD value:

$${c}_{i} = {argmax}_{c ∈ \{ 1 , … , {C}^{*} \}} ( {R}_{i , c}^{2} ) . \qquad ( 42 )$$

Afterwards, the CDB method collects all instances into the tuple c_{CDB} where the CoD value and the maximum frequency rise are both above thresholds ε_{R}² and ε_{f}, respectively:

$${c}_{CBD} = ( i : {R}_{i , {c}_{i}}^{2} < {ε}_{{R}^{2}} , {max}_{1 \leq j \leq {10}^{r} + 1} ( {f}_{i , j} ) - {f}_{base , i} > {ε}_{f} , i = 1 , … , M - {10}^{r} - 1 ) . \qquad ( 43 )$$

Similarly to the MDB method, identified chirps are associated with contiguous segments in c_{CDB}. In each contiguous segment, the identified chirp is assigned to the instance i that has the highest R_{i,ci}² value. The CDB method is illustrated in FIG. 24c.

### Chirp Detection Based on Assumed Chirp Waveform

In order to assess the performance of the two algorithms detailed above, the time-frequency-shape-based (TFSB) chirp detection algorithm was chosen as a reference described in (Eske et al, 2023). This algorithm is based on the chirp waveform function

$$φ ( \tilde{T} ; \tilde{α} ) = \frac{2 {e}^{\overline{α} \hat{T}}}{1 + 2 {e}^{\overline{α} \hat{T}}} , \qquad ( 44 )$$

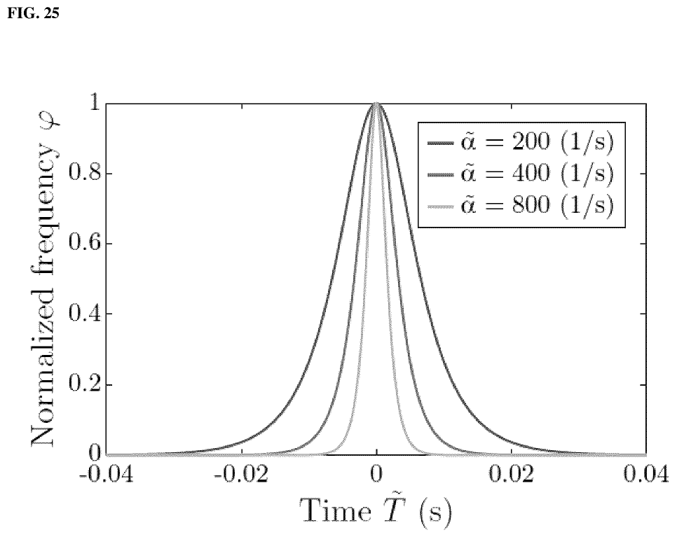

which is assumed to characterize, during chirps, the normalized frequency φ with respect to time \tilde{ }T. This function is parameterized by a single parameter \tilde{ }α that controls chirp duration (see FIG. 25).

The TFSB algorithm has 7 hyper-parameters, out of which 5 were fixed (see Table 9), and the remaining 2 were determined via cross-validation.

TABLE 9
Fixed hyper-parameters of the
time-frequency-shape-based
chirp detection algorithm
(for definition of parameters,
see Eske et al (2023))

| Parameter (unit) | Value |
|---|---|
| n_{wind} (1) | 100 |
| n_{med} (1) | 10 |
| n_{α} (1) | 200 |
| αₘᵢₙ (1/s) | 100 |
| αₘₐₓ (1/s) | 800 |

### Cross Validation

To determine the optimal hyper-parameter values hopt of detection algorithms, k-fold cross validation was used. In particular, indices i∈i_{GT} associated were randomized with time-series data segments Sᵢ and split them onto k number of equal-size folds: i_{GT,q}⊂i_{GT}, q=1, . . . , k. For each iteration step q=1, . . . , k, of cross validation, a single fold i_{GT,q} was used as a test set for determining the performance of the algorithm, while the rest of the folds were used as a training set. Note that only the two supervised algorithms were trained (for details, see Section 2.3.2), while the TFSB algorithm did not involve any training. The performance of each algorithm was determined by computing the false positive and false negative rates for each iteration step q=1, . . . , k, as

$${FP}_{q} = \frac{{\sum }_{s ∈ {1}_{{ax}_{g}}} {\sum }_{j = 1}^{{m}_{A , x}} 1 ( \vert \{ i : {\hat{T}}_{j}^{( R )} ∉ [ {T}_{i , 1}^{( s )} , {T}_{i , {l}_{c}}^{( s )} ] , 1 \leq i \leq {m}_{GT , s} \} \vert = {m}_{GT , s} )}{{\sum }_{s ∈ {I}_{{ax}_{g}}} {m}_{A , s}} , \\ {FN}_{q} = 1 - \frac{{\sum }_{s ∈ {1}_{{ax}_{g}}} {\sum }_{j = 1}^{{m}_{A , x}} 1 ( \vert \{ i : {\hat{T}}_{j}^{( R )} ∉ [ {T}_{i , 1}^{( s )} , {T}_{i , {l}_{c}}^{( s )} ] , 1 \leq i \leq {m}_{A , s} \} \vert > 0 )}{{\sum }_{s ∈ {I}_{{ax}_{g}}} {m}_{GT , s}} , \qquad ( 45 - 46 )$$

where 1(·) is the indicator function, Tⱼ^{(s)} denotes the j-th time instance of chirps detected by the algorithm in time-series data segment Sₛ, while T_{i,1}^{(s)} and T_{i,l1}^{(s)} correspond to the first and last data point of the i-th chirp sample in Gᵣ collected from data segment Sₛ. Parameters m_{A,s} and m_{GT,s} denote the total number of chirps detected by the algorithm in Sₛ, and collected manually from Ss, respectively. The overall performance of the algorithm was determined by averaging over all folds:

$$\overline{FP} ( h ) = \frac{1}{k} {\sum }_{q = 1}^{k} {FP}_{q} ( h ) , \overline{FN} ( h ) = \frac{1}{k} {\sum }_{q = 1}^{k} {FN}_{q} ( h ) . \qquad ( 47 )$$

Note that false positive and false negative rates depend on hyper-parameters h. The hyper-parameters were tuned, such that for a given maximum tolerated average false positive rate rFP, the average false negative rate is minimized, i.e.,

$${h}_{opi} ( {r}_{PP} ) = {argmin}_{h ∈ H ( {r}_{PP} )} ( \overline{FN} ( h ) ) , H ( {r}_{FP} ) = \{ h ∈ Ω : \overline{FP} ( h ) \leq {r}_{PP} \} , \qquad ( 48 )$$

where Ω is the search domain of hyper-parameters. At the maximum tolerated average false positive rate rFP, the lowest achievable average false negative rate is

r_{FN}(r_{FP})=F̅N̅(hₒₚₜ(r_{FP})). (49)

The implemented search domains of hyper-parameters are summarized in Table 10.

TABLE 10
Search domains of hyper-parameters for the Mahalanobis-distance-
based (MDB), coefficient-of-determination-based (CDB), and
time-frequency-shape-based (TFSB) chirp detection algorithms

| | Search domain | | |
|---|---|---|---|
| Hyper-parameter | MDB | ODB | TFSB |

| | | |
|---|---|---|
| N | {2, 3, . . . , 8} | n/a |
| C | {2, 3, . . . , 14} | n/a |

| ε_{f} | {0, 2, . . . 20} | |
|---|---|---|
| α | {0.01, 0.02, . . . , 0.15} | n/a |
| ε_{R}_{²} | n/a | {0.3, 0.35, . . . , 0.8} |

### Results

### Performance of Detection Algorithms

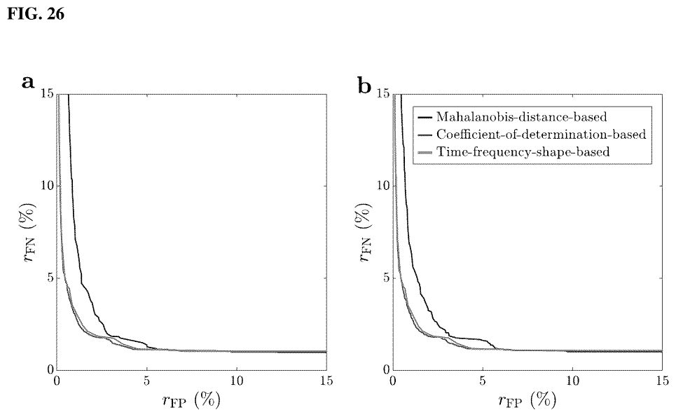

For the GT group G₂, the lowest achievable average false negative rate r_{FN} of each algorithm were computed at given average false positive rate tolerances r_{FP} (see FIG. 26) according to Eq. 49, using the search domains in Table 3. These results show that the performance of the MDB method is inferior to the CDB and TFSB methods. The CDB method performs better than the MDB and TFSB methods, although, the r_{FN}(r_{FP}) curves of the CDB and TFSB methods are nearly identical (FIG. 26).

### Principal Components and Explained Variance

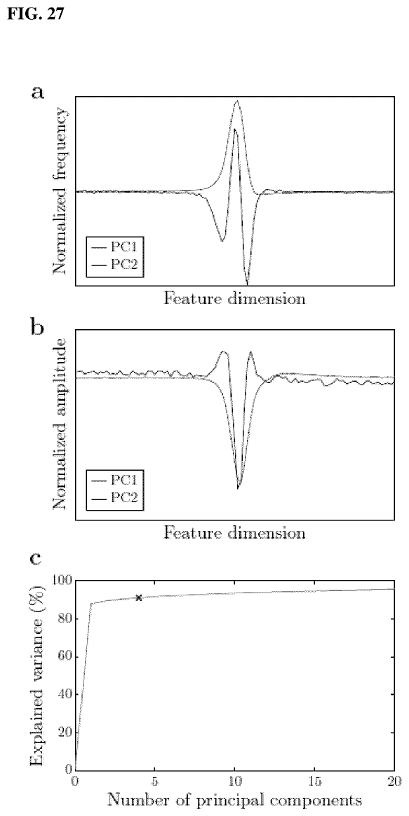

To illustrate waveform components that dominate GT group G₂, its PCs were computed (FIG. 27a,b) and the explained variance in terms of the number of its retained PCs (FIG. 27c). The first PC explains 90% of the variation in G₂ (FIG. 27c). The frequency shape of the first PC (PC1 in FIG. 27a,b) is similar to the chirp waveform of the TFSB method (cf. FIG. 25). This, together with the high percentage of explained variance associated with the first PC, result in a similar performance of the TFSB method and the CDB method (FIG. 26).

### Chirp Detection

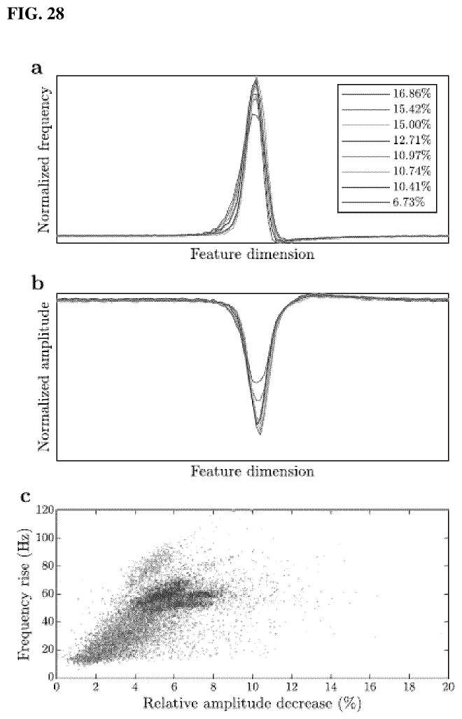

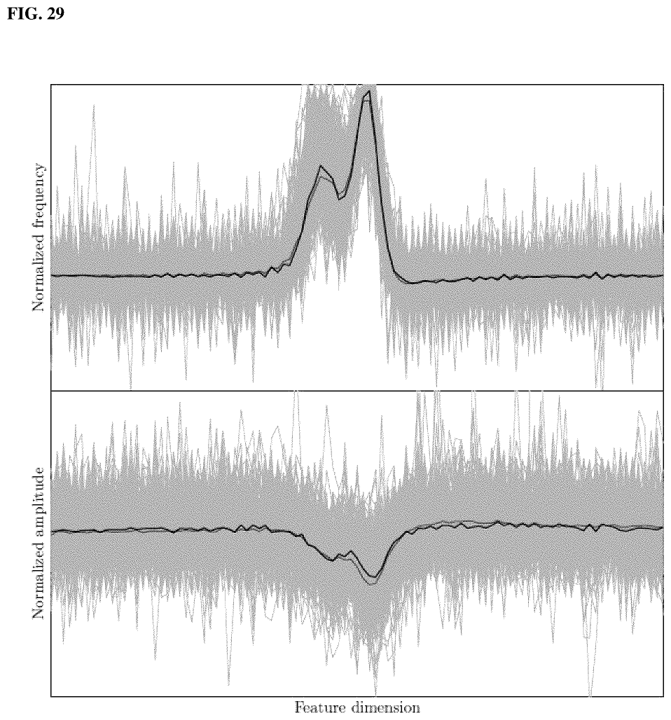

After cross-validation, a model was trined described above based on the entire GT data set G₂. An optimal hyper-parameters hₒₚₜ(r_{FP}=5%) determined via 4-fold cross-validation was used. The cluster means of the model, computed according to Eq. 32, are shown in FIG. 28. After training, the CDB method was employed (under hyper-parameters hₒₚₜ(r_{FP}=5%)) to detect chirps in all 1,680 min of EOD recordings. A total of 30,734 chirps were detected. All detected chirps assigned to the cluster mean with the smallest proportion (6.73%, see FIG. 28) were further investigated. To find sub-clusters, a new GMM was fitted on the these chirps according to Section 2.3.2 using N=4 and C=8. This analysis revealed a new chirp type, displayed in FIG. 29.

### Discussion

### Advantages of the Supervised-Learning Method

The results presented in this paper demonstrate the superiority of our supervised-learning algorithm over traditional methods for analysis of chirps produced by A. leptorhynchus.

The first advantage of our method lies in its versatility, compared to traditional approaches. As shown in Section 3.2, the TFSB method performs well for the herein analyzed signal segments because a single time-frequency waveform (associated with type-2 chirps) dominates the collected GT chirp data set, and this waveform matches well the assumed time-frequency shape. If multiple dominant waveforms are present in the GT chirp data set, or if the assumed time-frequency shape does not match the dominant chirp waveform, the performance of the TFSB method would be significantly worse. Furthermore, the design of a shape function representative of the dominant chirp waveform is rather cumbersome and impacted by the researcher's bias. In contrast, the supervised-learning algorithm autonomously trains chirp waveform models by fitting them to the collected GT chirp data. Given that the GT data set is representative of chirps in the analyzed signal, this algorithm provides an unbiased way for the automatic identification of dominant chirp waveforms in the signal.

The second advantage of our supervised-learning method is its ability to identify, in an unbiased way, possible sub-types of a signal. In the case of chirping behavior in A. leptorhynchus, visual inspection of time-frequency plots and time-voltage plots has suggested six subtypes of this signal. Although, in the present study, the analyzed recordings contained predominantly a single chirp subtype (type-2), our method suggested that further differentiation of this subtype is possible (see Section 3.3).

The third advantage of our method is that, compared to traditional approaches, it extracts more information from the samples used for the validation of the algorithm. Note that only a few traditional approaches validate their algorithm by signals with known chirp types and locations. However, these approaches use the collected set of chirps only to test efficiency, and thus the algorithm itself is not informed by the known chirp content. By contrast, our supervised-learning method takes full advantage of known chirps and utilizes them for both training the algorithm and testing its efficiency.

### Limitations of the Method

Although our algorithm trains itself and identifies chirp clusters automatically, it still relies on the collection of GT samples. Consequently, results are still impacted by the bias of the individual who collects the chirp samples of the GT set. This bias can be reduced if multiple individuals carry out chirp collection using the same signal, and if the GT set is assembled based on the overlap across sets collected by different individuals.

When chirps appear in the signal at a low frequency, the time needed for an individual to collect a sufficiently large GT set increases. While the validation of any algorithm requires the collection of all chirps from a test signal, the number of samples needed by our supervised-learning method is higher than the number of samples needed for validation only. Nevertheless, our method can still be advantageous compared to traditional approaches when already detected chirp types are expected in future experiments. In such cases, the cluster shapes from already collected GT sets can be reused. Furthermore, one can even build libraries of cluster shapes which can then be employed to “scan” signals for all formerly identified chirp shapes.

Our method is based on the time-frequency-amplitude signal (Tk, fk,Ak), k=1, 2, . . . . However, the method for the computation of this signal, described in Section 2.2, works only in the case of time-voltage data being generated by a single EOD source. For the analysis of multiple (either synthetic or recorded from fish) simultaneously recorded EOD signals, one must employ a different method) to extract individual times-frequency-amplitude signals.

### Perspectives

The presented supervised learning algorithm provides a valuable tool for further examining the function of chirps. In the present study, it has not only enabled us to validate the previous classification of chirps into different subtypes, but also suggested that further differentiation of these subtypes is possible. Whether these sub-subtypes of chirps subserve any behavioral function remains to be examined.

It is likely that other algorithms based on supervised machine learning will exhibit advantages similar to our approach. Thus, the present study might serve as proof-of-principle of the suitability of the supervised-machine-learning paradigm for a broad range of signals analyzed in neuroethology. It is likely that, in future investigations, algorithms based on machine-learning paradigms like the one implemented in the present study will increasingly become standard tools for signal analysis in neurocthological research.

### REFERENCES CITED

Allen K M, Marsat G (2019) Neural processing of communication signals: the extent of sender-receiver matching varies across species of Apteronotus. eNeuro 6(2):ENEURO.0392-18.2019. https://doi.org/10. 1523/encuro.0392-18.2019, URL https://www.eneuro.org/content/eneuro/6/2/ENEURO.0392-18.2019.full.pdf
Aumentado-Armstrong T, Metzen M G, Sproule M K J, et al (2015) Electrosensory midbrain neurons display feature invariant responses to natural communication stimuli. PLoS Comput Biol 11(10):e1004,430. https://doi.org/10.1371/journal.pcbi.1004430, URL https://dx.plos.org/10.1371/ journal.pcbi.1004430
Bastian J, Schniederjan S, Nguyenkim J (2001) Arginine vasotocin modulates a sexually dimorphic communication behavior in the weakly electric fish Apteronotus leptorhynchus. J Exp Biol 204(11): 1909-1923. https://doi.org/10.1242/jeb.204.11.1909, URL https://journals.biologists.com/jeb/article/204/11/1909/32945/Arginine-Vasotocin-Modulates-a-Sexually-Dimorphic
Bishop C M (2006) Pattern recognition and machine learning. Springer Science+Business Media, New York, NY
Bradbury J W, Vchrencamp S L (2011) Principles of animal communication, 2nd ed. Sinauer Associates, Sunderland, M A Dulka J G, Maler L (1994) Testosterone modulates female chirping behavior in the weakly electric fish, Apteronotus leptorhynchus. J Comp Physiol A 174:331-343. https://doi.org/10.1007/BF00240215, URL https://link. springer.com/article/10.1007/BF00240215
Dunlap K D (2002) Hormonal and body size correlates of electrocommunication behavior during dyadic interactions in weakly electric fish, Apteronotus leptorhynchus. Horm Behav 41:187-194. https://doi.org/10.1006/hbeh. 2001.1744, URL https://www.sciencedirect.com/science/article/pii/S0018506X01917441
Dunlap K D, Larkins-Ford J (2003) Diversity in the structure of electrocommunication signals within a genus of electric fish, Apteronotus. J Comp Physiol A 189(2): 153-161. https://doi.org/10. 1007/s00359-003-0393-3, URL https://link.springer.com/article/10.1007/s00359-003-0393-3
Dunlap K D, Thomas P, Zakon H H (1998) Diversity of sexual dimorphism in electrocommunication signals and its androgen regulation in a genus of electric fish, Apteronotus. J Comp Physiol A 183(1):77-86. https://doi.org/10.1007/s003590050236, URL https://www.ncbi.nlm.nih.gov/pubmed/9691480
Dunlap K D, Jashari D, Pappas K M (2011) Glucocorticoid receptor blockade inhibits brain cell addition and aggressive signaling in electric fish, Apteronotus leptorhynchus. Horm Behav 60(3):275-283. https://doi.org/10.1016/j.yhbch.2011.06.001, URL https://www.sciencedirect.com/science/article/pii/S0018506X11001280
Engler G, Zupanc G K H (2001) Differential production of chirping behavior evoked by electrical stimulation of the weakly electric fish, Apteronotus leptorhynchus. J Comp Physiol A 187:747-756. https://doi.org/10.1007/s00359-001-0248-8, URL https://link.springer.com/article/10.1007/s00359-001-0248-8
Engler G, Fogarty C M, Banks J R, et al (2000) Spontaneous modulations of the electric organ discharge in the weakly electric fish, Apteronotus leptorhynchus: a biophysical and behavioral analysis. J Comp Physiol A 186:645-660. https://doi.org/10.1007/s003590000118, URL https://link.springer.com/article/10.1007/s003590000118
Eske A I, Lehotzky D, Ahmed M, et al (2023) The effect of urethane and MS-222 anesthesia on the electric organ discharge of the weakly electric fish Apteronotus leptorhynchus. J Comp Physiol A https://doi.org/10.1007/s00359-022-01606-6, URL https://link.springer.com/article/10.1007/s00359-022-01606-6
Feng A S, Riede T, Arch V S, et al (2009) Diversity of the vocal signals of concave-cared torrent frogs (Odorrana tormota): evidence for individual signatures. Ethology 115(11):1015-1028. https://doi.org/10.1111/j.1439-0310.2009.01692.x, URL https://onlinelibrary.wiley.com/doi/abs/10.1111/j.1439-0310.2009.01692.x
Field C E, Petersen T A, Alves-Gomes J A, et al (2019) A JAR of chirps: the gymnotiform chirp can function as both a communication signal and a jamming avoidance response. Front Integr Neurosci 13:55. https://doi.org/10.3389/fnint.2019.00055, URL https://www.frontiersin.org/articles/10.3389/fnint.2019.00055
Gama Salgado J A, Zupanc G K H (2011) Echo response to chirping in the weakly electric brown ghost knifefish (Apteronotus leptorhynchus): role of frequency and amplitude modulations. Can J Zool 89:498-508. https://doi.org/10.1139/Z11-014, URL https://cdnsciencepub.com/doi/10.1139/211-014
Hechavarría J C, Beetz J M, García-Rosales F, et al (2020) Bats distress vocalizations carry fast amplitude modulations that could represent an acoustic correlate of roughness. Sci Rep 10(1):7332. https://doi.org/10.1038/s41598-020-64323-7, URL https://www.nature.com/articles/s41598-020-64323-7
Henninger J, Krahe R, Kirschbaum F, et al (2018) Statistics of natural communication signals observed in the wild identify important yet neglected stimulus regimes in weakly electric fish. J Neurosci 38(24):5456-5465. https://doi.org/10.1523/jneurosci.0350-18.2018, URLhttps://www.jneurosci.org/content/38/24/5456
Hupé G J, Lewis J E (2008) Electrocommunication signals in free swimming brown ghost knifefish, Apteronotus leptorhynchus. J Exp Biol 211(Pt 10):1657-1667. https://doi.org/10.1242/jeb.013516, URL https://www.ncbi.nlm.nih.gov/pubmed/18456893
IlieşI, Traniello I M, Sîrbulescu R F, et al (2014) Determination of relative age using growth increments of scales as a minimally invasive method in the tropical freshwater Apteronotus leptorhynchus. Journal of Fish Biology 84(5):1312-1325. https://doi.org/https://doi.org/10.1111/jfb.12354, URL https://onlinelibrary.wiley.com/doi/abs/10.1111/jfb.12354
Kolodziejski J A, Sanford S E, Smith G T (2007) Stimulus frequency differentially affects chirping in two species of weakly electric fish: implications for the evolution of signal structure and function. J Exp Biol 210(14):2501-2509. https://doi.org/10.1242/jeb.005272, URL https://journals.biologists.com/jeb/article/210/14/2501/16923/ Stimulus-frequency-differentially-affects-chirping
Meyer J H, Leong M, Keller C H (1987) Hormone-induced and maturational changes in electric organ discharges and electroreceptor tuning in the weakly electric fish Apteronotus. J Comp Physiol A 160(3):385-394. https://doi.org/10.1007/BF00613028, URL https://link.springer.com/article/10.1007/BF00613028
Neeley B, Overholt T, Artz E, et al (2018) Selective and context-dependent social and behavioral effects of Δ9-tetrahydrocannabinol in weakly electric fish. Brain Behav Evol 91(4):214-227. https://doi.org/10.1159/000490171, URL https://www.karger.com/DOI/10.1159/000490171
Raab T, Madhav M S, Jayakumar R P, et al (2022) Advances in non-invasive tracking of wave-type electric fish in natural and laboratory settings. Frontiers in Integrative Neuroscience 16. https://doi.org/10.3389/fnint.2022.965211, URL https://www.frontiersin.org/articles/10.3389/fnint.2022.965211
Schwartz C, Tressler J, Keller H, et al (2007) The tiny difference between foraging and communication buzzes uttered by the mexican free-tailed bat, Tadarida brasiliensis. J Comp Physiol A 193(8):853-863. https://doi.org/10.1007/s00359-007-0237-7, URL https://link.springer.com/article/10.1007/s00359-007-0237-7
Smith G T, Combs N (2008) Serotonergic activation of 5HT1A and 5HT2 receptors modulates sexually dimorphic communication signals in the weakly electric fish Apteronotus leptorhynchus. Horm Behav 54(1):69-82. https://doi.org/10.1016/j.yhbch.2008.01.009, URL https://www.sciencedirect.com/science/article/pii/S0018506X08000299
Triefenbach F, Zakon H (2003) Effects of sex, sensitivity and status on cue recognition in the weakly electric fish, Apteronotus leptorhynchus. Anim Behav 65:19-28. https://doi.org/10.1006/anbe.2002.2019, URL https://www.sciencedirect.com/science/article/pii/S0003347202920191
Zupanc G K H, Bullock T H (2005) From electrogenesis to electroreception: An overview. In: Bullock T H, Hopkins C D, Popper A N, et al (eds) Electroreception. Springer Science+Business Media, New York, NY, p5-46, https://doi.org/10.1007/0-387-28275-0 2, URL https://link.springer.com/chapter/10.1007/0-387-28275-0 2
Zupanc G K H, Maler L (1993) Evoked chirping in the weakly electric fish Apteronotus leptorhynchus: a quantitative biophysical analysis. Can J Zool 71:2301-2310. https://doi.org/10.1139/293-323, URL https://cdnsciencepub.com/doi/10.1139/293-323
Zupanc GKH, Sîrbulescu R F, Nichols A, et al (2006) Electric interactions through chirping behavior in the weakly electric fish, Apteronotus leptorhynchus. J Comp Physiol A 192:159-173. https://doi.org/10.1007/s00359-005-0058-5, URL https://link.springer.com/article/10.1007/s00359-005-0058-5
Zupanc G K H, Ilieş I, Sîrbulescu R F, et al (2014) Large-scale identification of proteins involved in the development of a sexually dimorphic behavior. J Neurophysiol 111:1646-1654. https://doi.org/10.1152/jn.00750.2013, URL https://journals.physiology.org/doi/full/10.1152/jn.00750.2013
Zupanc M M, Engler G, Midson A, et al (2001) Light-dark-controlled changes in modulations of the electric organ discharge in the telcost Apteronotus leptorhynchus. Anim Behav 62(6):1119-1128. https://doi.org/https://doi. org/10.1006/anbe.2001.1867, URL https://www.sciencedirect.com/science/article/pii/S0003347201918676

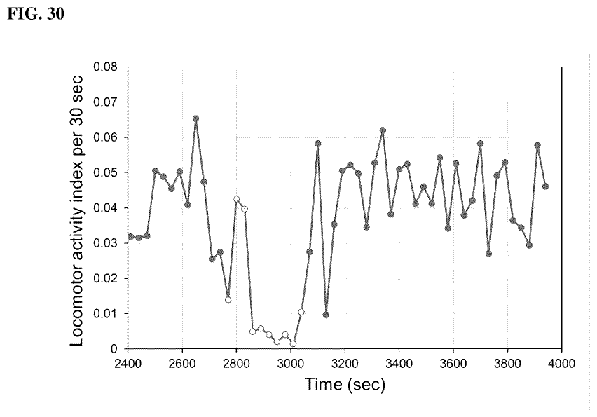

Neuro-Behavioral Assay extended to the monitoring of locomotor activity. The electric organ discharge (EOD) that is recorded as the assayed behavior contains not only information about the frequency and possible modulations of the EOD (‘chirps’) but also about the locomotor activity of the fish. The reason is that whenever the fish changes its relative position to the (fixed) recording electrodes, the recorded amplitude of the EOD is modulated, due to the vector-field property of the electric field produced by the fish (FIG. 30).

The unique feature that makes the Neuro-Behavioral Assay so powerful in comparison to other assays for testing of anesthetic compounds is that it assays a behavior that can be readily sampled (the EOD) as a proxy of the neural activity of the brainstem oscillator (pacemaker nucleus) that drives this behavior. This is possible because each volley of command spikes from the pacemaker nucleus results in one discharge of the electric organ, i.e., the frequency of the oscillation of the pacemaker nucleus equals the frequency of the EOD.

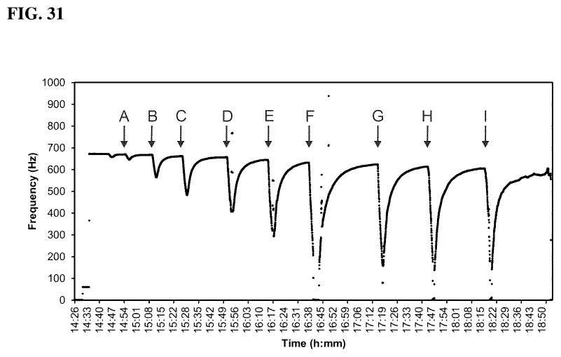

By performing extracellular recordings from the isolated pacemaker nucleus, and by exposing this in vitro preparation to various concentration of an anesthetic (MS-222), I have demonstrated that the effect of anesthetics on the EOD frequency (i.e., a dramatic drop in EOD frequency) is due to a direct effect of these anesthetics on the frequency of the synchronized oscillations of the neurons of the pacemaker nucleus (see FIG. 31).

Included in the characterization of the Neuro-Behavioral Assay is a negative control to show that the assay does not produce positive results in response to any neuroactive compound but that these results are specific to anesthetics and compounds with sedative effects. For this control, Amantadine was tested, which is used to treat symptoms of Parkinson's disease. This drug was testedat concentrations from 1 μM, to 5 μM, 10 μM, 20 μM, 30 μM, 40 μM, 50 μM, 60 μM, with exposure times of up to 48 hours. Despite the comprehensive testing, no effects of the drug in the Neuro-Behavioral Assay on (a) EOD frequency, (b) EOD modulations, (c) locomotor activity were observed.

It has been demonstrated that the Neuro-Behavioral Assay is not only applicable to screening of water-soluble anesthetic compounds but also to compounds with sedative side-effects, as found in a large number of over-the-counter drugs. Proof-of-concept was established by demonstrating a pronounced effect of the cough suppressant “RoboTablets” (the active ingredient is dextromethorphan) at a concentration of 0.002%. The following effects were found: (a) manifold increase in EOD modulations (‘chirps’); (b) enormous increase in locomotor activity, including rolling of body along the longitudinal axis. This demonstration is highly significant, as it indicates potential for tremendous broadening of the market for application of the Neuro-Behavioral Assay.

### INCORPORATION BY REFERENCE

All U.S. and PCT patent application publications and U.S. patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A system for evaluating water-soluble anesthetics, comprising:
a tank having a first end and a second end and at least one sidewall extending therebetween, the tank configured to receive water;
a tube disposed within the tank, the tube having a first end and a second end, having a cylindrical sidewall extending therebetween, the tube further comprising at least one end cap disposed at least one of the first end and the second end,
wherein the tube is at least partially submerged in the water within the tank and configured to at least partially house a subject;
a first electrode disposed at the first end of the tube, a second electrode disposed at the second end of the tube, and a ground electrode disposed within the tank; and
a computing device in electrical communication with the first, second and ground electrodes,
wherein the computing device is configured to receive electrical signals from the electrodes.

2-20. (canceled)

21. A method for evaluating water-soluble anesthetics, comprising:
providing a plurality of subjects in a community tank containing water, wherein each subject is capable of producing electrical organ discharges (EOD);
selecting at least one subject among the plurality of subjects;
introducing the selected at least one subject into a tube at least partially submerged in an isolation tank, the isolation tank comprising water of a first temperature and inorganic salts;
detecting a first frequency of EOD of the physiological subject after a first predetermined gap time has elapsed;
introducing the selected at least one subject into a compound tank having at least one compound diluted in water therein for a predetermined exposure period;
reintroducing the selected at least one subject into the tube submerged in the isolation tank;
detecting a second frequency of EOD of the at least one subject;
detecting a third frequency of EOD of the at least one subject after a predetermined gap time has elapsed;
introducing the selected at least one subject to a control tank having water for a predetermined control time;
detecting a fourth frequency of EOD of the at least one subject; and
transmitting the first, second, third and fourth frequency of EOD to a computing device.

22. The method of claim 21, wherein detecting the third frequency of EOD, introducing the selected at least one subject to the control tank and detecting the fourth frequency of EOD are performed prior to detecting the first frequency of EOD.

23. The method of claim 21, wherein the compound is selected from the group consisting of urethane, MS-222 (tricaine methanesulfonate or 3-aminobenzoic acid ethyl ester methanesulfonate), eugenol, and a compound having a sedative side effect, and combinations of any of them.

24. The method of claim 21, further comprising maintaining the first temperature within a predetermined temperature range.

25. The method of claim 21, further comprising measuring water conductivity of the isolation tank until a predetermined threshold water conductivity is achieved.

26. The method of claim 21, further comprising illuminating the isolation tank for a predetermined illumination period.

27. The method of claim 21, wherein detecting the first and third frequency of EOD comprises measuring EOD of the at least one subject over a period of 30 minutes.

28. The method of claim 21, wherein detecting the second and fourth frequency of EOD comprises measuring EOD of the at least one subject over a period of 180 minutes.

29. The method of claim 21, wherein the predetermined gap time is greater than or equal to one day.

30. The method of claim 21, wherein the compound tank comprises 2.5% urethane or 0.02% MS-222.

31. The method of claim 21, wherein the compound tank comprises between 30-60 microliters of eugenol per liter of water.

32. A method for evaluating water-soluble compounds , comprising:
providing a plurality of subjects in a community tank containing water, wherein each subject is capable of producing electrical organ discharges (EOD);
selecting at least one subject among the plurality of subjects;
introducing the selected at least one subject into a tube at least partially submerged in an isolation tank;
detecting a first frequency of EOD of the at least one subject for a first predetermined time period;
introducing the selected at least one subject within the tube into a compound tank having at least one compound diluted in water therein for a predetermined exposure period;
detecting a second frequency of EOD of the at least one subject;
reintroducing the selected at least one subject within the tube into the isolation tank;
detecting a third frequency of EOD of the at least one subject for a second predetermined time period; and
transmitting the first, second, and third frequency of EOD to a computing device.

33-35. (canceled)

36. The method of claim 32, wherein the compound is selected from the group consisting of urethane, MS-222 and eugenol, and combinations of any of them.

37. The method of claim 32, wherein the compound is diluted in water at a concentration of between approximately 30-60 microliters per liter of water.

38. The method of claim 32, wherein the compound iseugenol.

39-41. (canceled)

42. The method of claim 32, wherein the first, second and third frequency of EOD are continuously measured during the first predetermined time period, exposure period and second predetermined period, respectively.

43. The method of claim 21, further comprising analyzing the frequencies amplitude and frequency of EOD via at least one supervised learning algorithm.

44. The method of claim 43, wherein the supervised learning algorithm is configured to identify a subtype of the EOD.

45. The method of claim 43, wherein the supervised learning algorithm is configured to identify at least one of a time-series frequency and amplitude of the EOD.

46. The method of claim 43, wherein the supervised learning algorithm is trained with a ground truth data set, the ground truth data set that correlates time instances and subtype of EOD to frequency and amplitude of EOD.
